SEQUENCE LISTING (16 sequences)

SEQ ID NO: 1 - DNA, 20346 bp, Escherichia coli
cagaaggggaactggcatcgcacgatgccgcagtaaatgacgggaaacgcgatacgcgcaagacgtatcgcgttaggtcacaattagtgggaggctctgattgctgctttcaaggccggaagccgggacgggctggcactgagcttatcaatccccatggtgagcagtcgtgctgtttgctgggtatctccagccatttcgccgcacatactgaccggaatttcattattacgtccggcctgacacaccatagcaatagcgttaatcaccgcgtcattacgataatcgaccagtttcgcgacggtgctatttcctctgtctgccgccataatatattgcgtcagatcgttggtgccgatggaaaagaaatcgacttcactggctaatttttctgcaaccatgactgcagcaggaacctcaatcataattcccagcgccggaagattttctgcagtaagtcccagcagttttgcctgcgtttgtatcagcgttttgacggcgttgacctcctcgagcgttgaaaccatcggcagcataatttgcaacgttgggtattctttcccggcccggagaagtgcgcgtaattgcggaataaatatttgcggatgcgccaggcacaaccggataccgcgaagtcccaggaacgggttgtcctctgcgggtaaagggtacgaaggtaatggcttgtcaccgccaatatccagagtgcggatggtgactggtttatcgccaaaagcctgagcaacagaacaatacacgcgaaactgctcttcttcatcgggtaatgttgcagactcatggaataaaaactctgtgcgcagtaaacccacgccctcagccccgtgagatagcgcagcctcaatatcttgcaggtcaccaatattcgccagcacgctgatttttcgcccaccttgcgtgacagcgggcagcatggcatcagctaactgacgatccctttgttgccgccatgcttcggcgcgttgcagtaaatctaggcgggtgacctcatccggctgtagccacaaacggcctgtttcaccatcaagaatgaccgtgtcgttatcgcggacgttttgcggggtaatagctgccttcacaatggcaggaatacccattgctctggcaaggatcgctgtatgtgaagttgttccgccattatgaagacaaatgccaagaatgtgtgctttattcaggctggttatctgtgatggcaacagatcgttggctaacaaaatgcagggcgcggtagtgataaagtgttgttcacttaccccgcaaagctggttcagtacctgacgtgtaaggtcacggatatctgcctcgcgttcacgtaaatacagtgtttccgcctgacaatattgctctgctaatgcctgcatgacttctaaccatgcgaattccgcctgtttgccctttgccagacgttgctgtaattgccggttaatctctggatcttcaagcatcattttatgggcggcaaaaatatcgccttcagggcctgccgctttatcggcaagttcacaaacaacaatgtcgatagcagactcgattcgcgacagctcttcttgcggagaatcgacggtgcgctgcgtaatttctggtcgttcgcaggtaaaccatttaacaggaccaatagcgataccattttgtactgacaagcctgatattgcgccatcgacaggggcgctgaccaccggcgtttccgttagcggaatctctgaagaagcgagcaggtgagcttcaccaaaatgatttttcgccaacgtttcaattgccttgagagcgtcactggcatcggtgccttgggcctggaagatgatttcatcgccacaagcggacgcccagcattgccagttcgttgagactgcgggtgctggcgcgtttatcgcctttgatgacgataatgtttgccgaatatcgtgctgatactttcaggatgctggctgccggacgagcatgaatgccgtggggattacgtatcgtccagcaaaaagtttgccattcatcgcaatgattgactggtgatattactggaataacctcgcctgcagttgaggatggcgtgagttgctgctgtttagcacacagagcaagatccacttcagcgataatctccgtcaccgaaagccccgctgcggcagacgttaccgctgacatggcacctttcactacaggggcggcgagcagataaattgcacttctttgctcttcagaaagcagctccagcgccatctcggtactaagcagcgcactgcccatatccatgagtaccagaacgccatccggtgaccagaccgattcaatcgcagacataatggcaatagcatcagtgccaatcggattttgcggatcatcaatacctgcagcaatggccagaggaacgtcccctccagacatctgtagtgctaattcctcaaccccctgggcgagacgtagactatgagaaacaataacaatggcaaccataatttatcccgcgaatccttcctgcatagccttaatcatcagccaggaggaggtcgcgcctggatcctgatggcccacactgcgagcgccgagataactggcacgtccttttttcgcctgcatattgatggtgttatcgacggctgttgccgcatcttgcaccatcgtattcagcagaagggaaggcgacattccggcttgcaggttctttttggcttcgtggagaactggaacccagacatcacacagtgttttatccccttgttctgcttttcctcgtgcgataacgcctgcaatcccttcttccagacacgccagccactcttccagagttaattctgttctggctcccactgccgcagaagcgcgaataaataatgtgccgtaaagaggaccactggcaccgccaacgctggaaagtagtttcatgccagtatttttaaggatattgccaatatgctgacgttcaatgtcgggcaacgtcgctttcacggcactgaaaccacgattcatattgaggccgtggtcggcatcaccaatatctctgtcaagtgcggttagaaaatcttgttgttcagtaagatgtgcagcacaactctccagccaggcaataacatgtttgttttgaattttcatataatgtccttattaacatccccaacgaagcgctggcgtattgaccggggcatcccaaagttgcaaaaactctggggtacatttcagcaggctaatggaaaatccggccatatctaatgatgtacaataattaccgaccaactggcggctaattttaattccctggcgctgtgcggcgcacataaataccctggctacaccatataactctgattcaggcgtgctgccaagtccgttaattaacacaatatattcggcattttgatcgaatgactcattcattgatgatgcgtcaatccatccccccgcatggcgatcccaatggcgtaatgtgcgtcgccagggggtattatccagtaattgcgctatcacgctgtcgattaaagtgttcagatcttgcaaagtgcggcgttcaatgccgggttcaccgtggatcccgacaccaaattcgatttcattatcagccagttcaaatgagggttttcccgcggcgggaaccgtacaggctgacagtgcgatgccaaaagaacgggtggcattgttaacatctttgccaagttgctcgcactgccccaggttatatccctgaagtgcagccgcaccgacaatcttttcaacaaaaactgtacccgcaacaccgcgacgtccggctgtatacaggctatcttttaccgctacatcatcatcaataatgacggtcccaacggcgatgccttcttcgtgtaacatctcgactgccgtttcgaaattcagaatatcgccagtgtaatttttgatgaaaaacagaacgccagaaccattatcaaccgctttggcacattcgatcatttggtctggtgtgggcgatgtgaagatttcaccaggacatgcaccggttaacattcccataccgacaaaacctgcatgcagtggttcatggcctgagccgccgcctgaaatcagcgccactgcattatcggtttgtttgcaccacacatatcggggtgcgtaatttgcctgcaggtgtggccaggtggcgatgagtccttccatttgttctgtgacaacagaatcaatctggttaattaatttcttcatagtcattcctgttctgataaattacgtaaatcgcgctaaatatgcttgcgagattaattctccagccgcaataaaacaaatcgcacgataataagattcataatgaaacagttaattcattgtgttgttccaaaatgaaatgggatcggtgacaagattaggggtttgtgattaaagcataaaaaatatttttattattgctttgggatgatgttttttacagcgcgatcataaaacataatttttcgcgaatgaaatgtttgccgccactggatgcaacgagtaggttaacagtgaagcaaaacaagttacgcaaggaatgatatggataagattattataagtccggctaaatatatccagggaaatggttctctggataatattgctacatatgcggcgagtttgggaacagaaccattaattattgcagatgaatttgtgaccggtctggttggtgacagagtaagccagagctttgcaagagaaaacattattgcggattttgatgttttttgcggtgaatgctcgcaaaatgaaatttctcgaattcgtaagaaatttaatcagcgtaaatataatgtcgttattggtattggcggcggtaaaacgttggatacggcaaaagccgtagcgtattaccaaaaaattccggtggtggtggtgccgacaattgcttccacagatgcgccaaccagttctctggcagtaatttacaccccggatggtcagtttagcgaatatctgtttttcccgaaaaacccggatatggtgattatggatactggcgtcatttcagccgcgccagtgcgtctgctggtggccggtatgggagatgcattgtcaacctggtttgaggcgagagccaatcaggcgtcaggaaaagccacaatggcaggtggggcgtcgacactggcggcactggctattgcccgactctgttataccacgctactggaagatggctataaggcaaaagtcgcggttgaacaaggtgttagcaccaaagcggtagagaatattatcgaagctaatacctatcttagcggcattggatttgaaagtagcggcctggcggcagctcatgccattcataacggcatgacgcaactggaggaatgtcatcattgctaccacggtgaaaaagtggcatttggcgtcctggtacagctcgtactggaaaacgcaccacaggaggaaattgaaaccgtactcaatttctgccatagcgttggcttgccgacaaatcttcatatgttaggcgtgaaggaaattaataaagataaattacgggcagtggctaaagccgcgactgctgaaggggaaactatccataatatgccattcgtagtaacagcgcaggacgttctttgtgcaattttaactgcacatcatttgggactttaaaaaataatacggcctgtgcaaagcaggccgttggtgtcattttaaatacttcattcgggaataaatatgaccagtaactggaaacgttggatcacgctggcgttatgtggtattgccggaagtgcaatatataaattgccttatttacgggaaacctattacgacgcgatgcagcaggctacaggagcgactaatgctgaacttggttttctaatgacagcctatggtctggtgaactttttattatatctacctggcggttgggctgccgatagattttctgcacgtaaattaatgaccttttcattaatttccaccgggataagcggtttttattatgcgaccttcccctcgtatactatgatctgtttgttacatgcactttgggctgttacaaccgtatttacgttttgggccgtgtgtgtgcgaattattcgtaccctgggaaccagcgaagaacaaggacgcttatacggttactggtttctgggtaagggattaacctccattgtactgggatttctttccgtgccggtatttgccaaatttggcgaaggtgttgatggtttacgggcgacaattattttttattccgtagtgactattctggccggtgttttagcctggtttgtttgtcaggatgaaacgcacagcgaagataaagcgaatttccgtctggcagatatggcattcgtgctgaaaatgccgacggtttggctggcaggtgttgtgaccttttgtatgtggtcaatttatatcggctttggcatggtcacgccgtatctcacgcagatcctgcatatgggcgaatcggaagtggcagttgccagtattcttcgcgcctatgtgttatttgcgatggggggattaattggcggacaactggccgaccgctgtgcgtcacgcacgcgttttatgatttacgcctttatcggcatgattgtctttactaccgtttatttcttcctgcccggtgagtcgcgctacgtcactattgcgctggcgaatatggttgcgttgggcgtgtttatttactctgcgaatgccgtgttcttctcaattattgatgaggttcggatcccggcaaaagtcacagggactgctgcgggtctgatctctttactgacctatttcccggaaatctactgctacacgatggtgggcaatatggtcgaccgcaaaccgggtattgccggttatcaggatgttttcctgttcatgttggtttgcgcattcatcggccttatcgccgcactggtcctccagcgtgtgaatcgtaaaagtaaaaatcaagtcaatgaaatcactcaaaacaatgcggattgcgcatcctgaatctgacgggggaaccaatggtggacatgattaatgaaagtgcacggcaaacgccagtcattgcacaaacggacgttctggttatcgggggcggtccggcaggattatccgctgccattgcggcagggcggttaggtgccagaaccatgattgttgagcgctacgggtcgctaggcggcgtattgacgcaggtcggggtagaaagttttgcctggtatcgtcatccggggacggaagattgtgaagggatctgtcgtgagtatgaaggccgcgcacgagcgctgggtttcacacgaccagaacctcagtcaattagcgaagttatagatactgaaggatttaaagttgtcgccgatcagatgattacggaatctggcgttgagccgttatatcactcctgggttgtggacgtgatcaaggacggggatacgttatgcggtgttatcgtcgagaataaatcaggtcgaggggcaattctggcgaaaagaatcgtcgattgcacgggggatgctgatattgccgctcgtgcaggcgcgccctggacgaaacggagcaaggaccaactgatgggcgtcaccgtgatgttcagttgcgcaggtgttgatgtggcacgctttaaccgttttgttgcggaagaacttaagccgacctacgcggattggggcaaaaactggacgattcaaaccacgggtaaagaagacccgatgtttagcccgtatatggaggatatttttacccgcgcgcaacaggatggtgtgattccaggtgacgcccaggcgattgccggaacctggtcgaccttttctgaaagcggtgaggctttccagatgaatatggtgtacgcctttggttttgactgtaccgatgtcttcgatttaaccaaagctgagattgccggaaggcagcaagcattatgggcaattgacgcactacgccactatgttccgggctttgaaaatgtacggttacgcaattttggtgccacgctggggacgcgtgaatcacggcttattgagggggaaatacgtattgctgatgattacgtccttaatcaggggcgttgttcggacagtgtagggattttcccggaatttattgatggttccggttatctcattttgccaacgaccgggcgtttctttcagatcccttatggttgtctggtgccgcaaaaagtggagaaccttttggtcgccggtcgctgtatttccgcaggcgtagttgcacatacttctatgcgtaacatgatgtgttgtgccgttaccggtgaggccgcaggtactgccgccgtggtttcgctacagcaaaattgcaccgtgcgtcaggttgctatccctgatttgcaaaacacgctgcaacagcagggcgttcgtctggcataacaacaaggcgcagagagctgaaagcctgtctgcgccttgcaggagaaagaccatgagggatgatgacctggagcacttaagtgccaattactggataaccacagttctctgcggcctctttttggggatattcctgttcatgctgcaacagcatcagcgtggtttagctctgtattgtattgcggctctttcacttatttgcgcctttttttctttacgcctttcccgacaattgcaacacatgaaaaaggaacggcgaaatattcgacctcattgaatgaaaacattcaggagactgaaatgcactatttaaaaaaacgcttttctttgattgaactgaatgtgtttatcccagcaatattatttattgcggtaatcattctgtgtttgacgatatatccacaagataccagccggtatataaataaaatacatcattttttaacctgggaaatgggcgggatttttctggtcatgacttttctggttgtactttgttgtctgtggctggccttctcacgttatggcgatatcttgttaggtcagtcgggagaaaaacctgacttcagcttgttaacctggcttggacttatttttacttctggaacggggggcagcttgttatatctggcctctgtagagtggatttggatcattcagcaaccgccttttggcgcaacagcaggaagcgctcaggctgctcgttgggcctctgtttatggcatgttccattgggggccgtccgcatgggcatggtatttgatttgcgccgttcctattggttggtttatgcatgttaagaaaacaaactcattaaaggtcagtgatttatgtcgtggatgtttgggagcgcgtgcggatggtttttgcgggcattgtgtgaattttttctacatgtttggtttgctcggcggcgcggtaacgtctctggcgctgggaacgccgatgatttctgccgtattttgccatgtgttccatctggatcctgccgggcagtttatcaatgtcatggttatttttatctggacgctagtaccattattcattctcttttttggacttaaaaaaggtgtggcatgggccagtaactggaatattcgtgctgacattttgatgctactggcaatactgatttgtggaccaacggcatttatacttaatcaatcaattgatggtctcggcctgatgctgcaaaattttgtagcgatgagtttaagtaccgatgctattggtcgtagcggatttccacagatgtggaccgtattttatttttcatggtgggtcgtgtatgccatcccattcggtttatttatcgcccgtatttcaaaaggaaggacgatccggcaattgattgtatgtggaactctggcaggttcattgggatgcatggttttttacatggtactggctaatttcggcttatcgttgcagacaactcatgtcatcgattttgttcccatacttaacgaacaagggcgaggcgttgtcgtttctcgtttactggagcagctacccgcaagtcaggtgtttttggttgcttttggggctatagcattaatttcatatattaccggacactgtactgtgggttatgccctcggttttgcgacgcaaaaacggccagataagtaaaagtgaaccggcattctggaacgtggcattttggttgattatgactggaatcgtcgccatcacactctatcttcttgatgcgcaaagtctgcaaccgctacaaacggtgtctattctggctggactaccgctttgcggcgtggtgtttattttattgaagagttttttgacgcagcttgcggctgaagagaaaaacgcgagagatgaataatgatgttcaggaatgcccggtaatttgcattatcgggcattttaccgaacgggaattaactcaggatcgcacgaaaatatcccgccagattttctccaagacgaattaaattattttctccctgactcagcgcggtcggtaaattgcaagggccgttgacgacagaaaaacaggcttctattccacactggcgtaactcattcagatcaagttgatcatcgatatacccggcaattgcgataaccggaacgcccatcatctgggcaactttggctgcgcctacgggcgctttgccattcaggctctgactatctaatttcccttcaccgacaatggccaatgctgcgtattttaaatgttgggtatattgaatcgcttccagcacgagcgaaatacccggttgtaagcgagcatcgagaaaagccatcaaggccgcgccaacgccgccagccgccccagaaccgggtatcgtatcgacgtctttaccacaacaacggtagatgcaacgggcatagtttttcattccctgttccagttgctcaaggtcgttagcactggcccctttttgttgagcaaaaacccgggtcgcgccgttatcgccggttagtgggttggtgacatcacaggccgcaataaatttcacatttttcaatcgtggatcgaggtgctgtaagtcaatgtggtgaattgccgccagccccatcccattaacagagactgatagcccttcggcgtctgtaaacgatgcgccaagtgccgacatcaacccaataccgccatcatttgtagcactgccacccaggcatagaataacggtatcaatattgtgttccagggcggcgagaataagttcgccagtgccaaacgacgtcgtataaaggggagaacgctggacaggagaaaccagttccagccctgaagcttgtgccagttctatcactgcggtggtttggttgcctaaaataccgtattgcgcatgtattgattcgccaagcggtccggtgacttttttagtgtaaaacgtaccgtcagtcgcatgaatgagtgtttcgagcgtgccttcgccgccgtcagccacgggcaaacacaccagttggctgccaggcaggaccttgcgccagccggttgccagcgcttcagcgactttccatgcgggaaggcattctttaaaggagtcgggggagataactatcttcatagcaggtctctcaaaataggggaatggggcagggcaatgagtgaacgaagggaaccggtggttcccttaattcattatttacggatagtgaaattggcttgtgtttcaagcgcccgaattaacgcagaatgatcccactctttaccaccgagcgcggcgcaggcgctaaataactgctgtgtcgtcgccgtgttaggaagcggggtattcaacaggcgggcattctccagcgccagattcagatctttttgatgcagactaatacgaaaaccgggttcgaatgttccttttatcatccgttcgccatgaacttccagcacgcgagaggaggctaaccctcccatcagcgcttcacgtacgcgggcgggatcggcacctgatttactggcaaagagcagggcttcagccaccgcttcaatattcagtgcaacaataatttggttagcgactttgcacgtttgaccatcaccgacgttaccgaccagagtgatatttttacccatcaattccagaatcggtttgatctgcaaataaacctcttcacatccgccaaccataatggaaagcgtcccggctttcgcgccaacttcaccgccagagacgggcgcatcgatatattccgcgcctaccgcgataatacgttgtgcaaatgctttggtggcaaccggggaaattgaactcatatcaatcacaattttcccgtggcttaaaccgtgtattacgccatagttgggatgaaaaagaacatcctcaacatgtggggtatcagggagcatggtgatgatgacctcacactcctgcgcgacttcacgcggtgtagagcaaacaatgccacgttcaccaataaactcttgcggtgcaggttcaaaatgcgctgaaaaataaagagtatgtccggcttgttgtaaattataagccatcggcttacccattatgccggtgccgataaatccaatattcatgataaatacctcagggattaaaattgagtctcgttttttagccagcgtagactttcagtcgttgtcgtagatggaacatattcgcaaccaatccatccctgataaccaatctggtctatataatttaacaaccacgggtaattaatttctcccgtccctggttcatgacgcccagggttatcggctaattgaatatgttcaatgttattcaggttgtttttgattgttgtagcaatattcccttccatgatttgcatatgataaatatcgtattgatagcgaaaatcaggatgatttacatcataaataatattgagggcttgtctggtgttgttaacaaaaaaaccgggaatatcttttgtattaatggcttccagaactaactttatgccatgagaagccaatttatctactgcatagcgcaaattttcgaccagtgtttcatgacattgttcatgcgaataaccacctggatgttttcctgcaaggcaattaacttgtgaacaattaagggcctgggcatattctatcgctctgcaaacaccctcctgaaattcttttgcccgactaggatgacatgcaataccccgttcaccctcttgccagttacccgcaggaagattaaacaaaacctgagtcagattatgttgatgcagttgcgaagccagctcgtcagtggaaaagtcatagggaaaaagatattcaacgccgtggaatcctgcgtgacttgcctgcgcaaaacgttcaataaatggatgctcggtaaaaagcattgataaattcgctgcaaactttgccataatcctgtttccttataaatcgagaatttcttcaaattctgtgactttattaatgtcaggtcccatagcgatattagtcacgcgttcagtaataatttctacaacaacaggtacgtgaaattcgtcacgcaaacgctgggcttcttgcagtgcaggcgcaatatcctgtgatgcgaatacgcgaatcgctttacatcctaatccttcaaccacagctttatgatcaacgccataaccattaatttctggagcattaatattttcaaatgacagttgaacgcaataatcaatatcaaatgcacgttgtgattgacgaatcaaaccaagataagcgttgttaagtaaaatatggatatacggaagattaaattgtgcgccgacggctaattcttcaatcaagaattgaaaatcgtaatcaccggatattgccacaacgggaacagatggatcagcttttaccgcgcccagtgctgcgggcattgtccagcctagcggcccagcctgacaggcattaatccagtggcgtgggcggtaaacatgtaaaaattggtttgcggcgatttgcgccagaccaattgtcgaaatatagcgtgtctccggtccgaaaaccttattcatttcgtgatacacgcgctgcggtttaattggattgcagtcaaagtctgaacgacgaagcattgtgcgtttacgctcggcacattcggcaatccagcgactgcggtctttcagttctccacgtgacttcatatcacgggcgacctgaataaatagcgttaaggcgctttcagcatcagaaacaatgcccagatccggcgcaaatatccggccaatttgtgcaggttcgatatcaacatgaatgaatttgcggccttcggtgtaggtctcaatggctccggtatggcggtttgcccaacggttaccgataccaaaaacaaaatctgaggccagataactggcgttaccatagcgatgacccgcctgacatcccatgcgaccaatcattagggggtgatcgtctgataaagcgccccaaccgcgtaaggtttgaatgacgggaacgccagttaactcgacaaactcacgcaacaattcactcgcttcggcgttaataatgccaccgccagcgatgatgacgggcttttcggcgtcatttagcatttctaatgcgcgtacggcctgggcccgtgttgccttaggttgccaggggattagtggctgatagagatcgatatcaaactcaatttcggtcatctgaacatcaaaagggagatctaaaagaaccgggccagggcggcctgaacgcatttcccagaaggctttctggaatatccccggcaactgacccgcttcgagaattgtgcgagccattttggttactggcgtggcgatagcctcaatatcgacggcctggaaatcttctttatgaagttttcctacgggagcttgaccagtaatgcagagaataggaatggaatccgcagaggcagaatacagcccggtaatcatatcagtgcccgcagggcctgatgtgccaatacacacgccaatattgccattctgactgcgggtgtaaccttcagccatatgcgaggcgccctcaacatgacgggccagaatatgatcaataccgcctgattttttcatcgcggcatagagtgggtttattgctgcgccgggtacaccaaaagcaacgctaatgccttcttttttcagtatttctactgcagcttcgattgcacgcatcctcgccatacattatttctcctgatatttacgctgagttgaatagtcagtcaattttggaatttaacgatgaaatatctttgaggaaataaaagggcagaacaaatttcgctgcactgtttcacagtgaaacaactcgtatgcctgtgttgtttcagtttgaataattataaatatcaaacgcaaagatttcagatgttgttggcagttttaaaatttatctaatgtatcattcgttttttattgctttaacggttattatttctaatgtatgtgtgattacattgttgataaggtctgtttttgtaatgatccaggctagatattattgcgtttgatcaagcttactggcatagcattctgatacaagttctgaaaatgacttgaatggatattattataatgaataaagagtcgtatcataatgatctaaaaaataaatggaaaatgtttgttaagcatggttgggtcgcgacaaattcgactaatcatgtgatgctgaggtcatggcaaaaatgtctcaagcattgtgaccctcgccactggaacacgcctgtcaaagcttccgggcaaacattgcagacgattttttctcgtaacgaagagtttattagaatttcacaaagagttgttgaagaccatttcaccctggctggtgacgaccggttggcatttttgatcattgatccacacggctgggttctatcgttgaatgcagcaggcgattattccagtcaattgcgagagttaggaattgaatctggtatgtcatgggccgaagatggcattggaaccaatgtatatagtctttgtcgggaaacaaatttatatacacaactggaaggagcagaacattttagtgaacagctacattgttatgcgatgagcgctgctccggtcattgattattatggtaatatacatggatacattgtatgtataattgaaaccaccgccgagcttgttaaattaaaaacatcatattcatgtgcaacagaaatcgctaattatatctatattgagaatgaacaaaagttaataaacaaagtactttgccagcataatgctgtgattgaatgtatggatgacggttttatttgttggaatagtcattctttaattacgatggttaattctcaagcacaaacattactgaatattgataaagaaagtttaattggtcagaacatccgaaaaggattcgtatttccgccgattttgaatgagggaattacacaacgaaacaaactatcgcaaaagcaaattgtcctcgaatgccgtggcgaatttattgaacttatggttacccttcgcccgttaagcgatggttcgtttttgctttttcttcatccattagacaaaatcaggaaaatagcccaacagcaaataagcactaatgcaaattttacctttgacagtttacatgcggcttcaggtggtatgaagcaggtattacttatcgctcgccgggcaattaaatccatctctccgattttgatcaatggcgaagaaggtgtgggaaaattgagtttggcgatggcaatacataatgagagcgagcaacgtgatgggccatttatttctgtagattgtcagatgctatcaccagaaaatatcttacacgaacttcttggctctgatgttggcccttcgccatcgaaatttgaactggctcataatggcaccttatatctggataaagtcgaatatctatcaggggaagttcagagtgttttattgaaagtattgaaaacggggcttgttactcgctcagacagccatcgtttgatccccgtacgctttcgtctgattacatgtaccagtagttctttacgtgagtacgtgcaacaaggggcttttagccgacagctatattatgaggtctccatgaatgaaattgaaattccgccattgcgcaaacgtcgtgaagatctcaagcaaatgattgacgatattattgataagtatcaggagcgcacgcgaaaaaaaatgacaatcacgcctgacgcaaattcagttctgcttgagtaccgttggcctgggaacatctccgagttcaaaaatcgaatggagaaggtatttattaactgcaatcgacttgtactcgggttagagaatattcctctggatatccgacaaaataacagtagtggcgacgatgatatccctcatcttacttcactggcagaattggagatgcaagctattgcgcatacatgtcgtgtctgcgaatggaatctaactaaagcagctgaagattaaaaattggtcgtacaacattatggcgcaaacttaagatctataatctctatccaaatgttgagcatgcagaattgatgcgttttcaaattgaaagaatttaatatcgcagtgtttcattttgatgcagtgctttaatggtattgaaatattgtgatgtgctccataaatgcccaatgtggttattagccagggagacggaaaatgtgtttacactccctgaaataataaaaaacaggcaaagagagatgatctcccttgtatgtctgtattagcatgatgttgcttgttaataattaaaaatatcaacgcgcttatataagcgcgggggattgttttactcaattattgaatacggagataaagtatggaattttatctggaacccgctcgtaatatacctgtactggcaaccacggaagtgttagttgttggtggtggtccatcgggtattgccgcagcaatgagtgccgctcgtgaaggcgcagctactatgctgattgaacgtttcggttgttttggcggaatgatgacaacggctggcgtcgagtcaattgcctggtggcgtcatgaaaatacggtagagtcaggtggactggcacgcgaaatagaagaaacggcaaaatcaatgggggcgtccagccctgagccgcaatcgaatagtcaggctattaacgaagagcgtttcaaactggttgcggatgcaatgcttgaacaggcaggtgtgcgccgcgtactacacattaccgccgttgatgttatcaagcagggcaataatttactcggcgtaataacagagagtaaatctggtcgtcaggctattttggcaaatgtcattattgactgtactggtgatgctgatattgcatggtttgccggagcaccatttattaagcgtgaacgcgaagagctaatgtgtatgacaaccgtttttagttgcgcaaatataaataaaaacgcgttcatgcaaaatattaatagcacggaacctaaatatggagactggggggcggatgaagaaaataaaaactggtcttatgatgttcatgaatcttgtcgcgatatgtttagcccttatctgggtaaagtcttgcgaaaggaaaggtcggcaggaattattccaaaagatgtgacgttaggcggttcctggagtacggtcaccgagtatggtgatgcgaattacttgaacgttgtcagcatccctgccgtcgattgtacggatgtttttgacctgacgcgtgcagaaatagaaggccgcaagcaagccatgcaggcgattgaagcgttgcgtcaattccagccaggatttgaacaggcacaattaaaaaatttcggtatgacggtgggaacaagagaatcaagacatattattgggcgagtccagcttacggaaaatgatatttgtaatgagggacgtcatgcggattcaataggggtattccctgagtttatagatggaaatggtcatcttaaattacctcttgaagcgaactattttcaaatcccttatggcgtaatgattccgcagcaagttgaaaacctgttggtttgcggacgggcaatcgatgcagataatttcgcctatgcgacaatccgtaatatggggtgttgtatggtcactggagaaggtgcagggactgccgctgctattgccattaaaaataacactaccgtttcacaggtagatattcagacggtacaggaacgcttacagcaaaatggcgtaaaagtcttttaacagggaataaagttatgaaagaaaaacccagtttttatgtcgcgcttacaccgatcatttttatgatgatcgtgttggttgttggcatcggtgttatgggctggcctgccagcgtctgtttattaatttcagcggcgttttcctgcattattgcaatggctaagctgaaatatacctgggatgaaatccaggggtttattatcgataaaatttcggcggtgatggcaccggtacttatcgttattttcgttggctttatggtcgcgacatggagctatgcaggtacactgccgatgctggtttattacggtatgttactggttgcgcccgcatggctttatgccatagcgttttttctgaacgcagtattgtcctatgtttccggggcatcatggggatctgtcgcttctattggtgttgcattaatgggcatcggttccggcttacatgccgatcttccaattctggcggcagccattgttaccggggcttattttggcgacaaactttcaccgttaagtgataccaccaacctgacttcagcggtaaccaaaactaaactttatgatctgattaaatatctactctggacgaccatcccatcaacgattattagcctgctgttttttacctggctgggaatgattcatacttctacgggctctatttcaccagaaagcatgcagatgctggtacagctcaaagagttatataagtttggtctgttaccgctactgcctatgcttattgttctgtgctttacctttttgcgtttgcctacagtaccggcattattaagcagtgcgttcgtggccgtattggttggatggctgtatcaaggtttcccgctggccgaaggtattaaggccacaatgagtggtttccgtctgacgatgatcactgattcaacgacgctgttaccttctgtgaaagaactgcttgaacaaggcggcctgaataatcagggcggattcctgtcatttattatctgcgccatgatgtttgctggcatcctgacgggaacaggaatgatggatgtcacattacaaagtgcggcaaacaaaataaaaagtgcttttggcgctattctggcaagtggtgttttagcggtcatcattaacttgcttaccggttctgatggtttgaataagattgttatatctgaattgatgatgaagaaattcgaagatcttaatttatctccattagttttagcccgaacgctggaagattttggcaccatgagtgcgccaataattccgtggtcagccgccggattgtatatggcgacgacgctaggtgttccgacgtttagttacctgccgtactgtgtattctgtttctgctcgatgatatttgcgttgatctatgcttctacaggtttccgtttactgcgcttcaacgaagcaaaggcctgagttattcaagataataaatgcgtttgccggatggattaatctccatcccgcgttaatgtcaaaattccatgttctgcagaat

SEQ ID NO: 2 - protein, 210 aa, Escherichia coli
MKIQNKHVIAWLESCAAHLTEQQDFLTALDRDIGDADHGLNMNRGFSAVKATLPDIERQHIGNILKNTGMKLLSSVGGASGPLYGTLFIRASAAVGARTELTLEEWLACLEEGIAGVIARGKAEQGDKTLCDVWVPVLHEAKKNLQAGMSPSLLLNTMVQDAATAVDNTINMQAKKGRASYLGARSVGHQDPGATSSWLMIKAMQEGFAG

SEQ ID NO: 3 - protein, 536 aa, Escherichia coli
VVSAPVDGAISGLSVQNGIAIGPVKWFTCERPEITQRTVDSPQEELSRIESAIDIVVCELADKAAGPEGDIFAAHKMMLEDPEINRQLQQRLAKGKQAEFAWLEVMQALAEQYCQAETLYLREREADIRDLTRQVLNQLCGVSEQHFITTAPCILLANDLLPSQITSLNKAHILGICLHNGGTTSHTAILARAMGIPAIVKAAITPQNVRDNDTVILDGETGRLWLQPDEVTRLDLLQRAEAWRQQRDRQLADAMLPAVTQGGRKISVLANIGDLQDIEAALSHGAEGVGLLRTEFLFHESATLPDEEEQFRVYCSVAQAFGDKPVTIRTLDIGGDKPLPSYPLPAEDNPFLGLRGIRLCLAHPQIFIPQLRALLRAGKEYPTLQIMLPMVSTLEEVNAVKTLIQTQAKLLGLTAENLPALGIMIEVPAAVMVAEKLASEVDFFSIGTNDLTQYIMAADRGNSTVAKLVDYRNDAVINAIAMVCQAGRNNEIPVSMCGEMAGDTQQTARLLTMGIDKLSASPSRLPALKAAIRASH

SEQ ID NO: 4 - protein, 214 aa, Escherichia coli
MVAIVIVSHSLRLAQGVEELALQMSGGDVPLAIAAGIDDPQNPIGTDAIAIMSAIESVWSPDGVLVLMDMGSALLSTEMALELLSEEQRSAIYLLAAPVVKGAMSAVTSAAAGLSVTEIIAEVDLALCAKQQQLTPSSTAGEVIPVISPVNHCDEWQTFCWTIRNPHGIHARPAASILKVSARYSANIIVIKGDKRASTRSLNELAMLGVRLWR

SEQ ID NO: 5 - protein, 356 aa, Escherichia coli
MTMKKLINQIDSVVTEQMEGLIATWPHLQANYAPRYVWCKQTDNAVALISGGGSGHEPLHAGFVGMGMLTGACPGEIFTSPTPDQMIECAKAVDNGSGVLFFIKNYTGDILNFETAVEMLHEEGIAVGTVIIDDDVAVKDSLYTAGRRGVAGTVFVEKIVGAAALQGYNLGQCEQLGKDVNNATRSFGIALSACTVPAAGKPSFELADNEIEFGVGIHGEPGIERRTLQDLNTLIDSVIAQLLDNTPWRRTLRHWDRHAGGWIDASSMNESFDQNAEYIVLINGLGSTPESELYGVARVFMCAAQRQGIKISRQLVGNYCTSLDMAGFSISLLKCTPEFLQLWDAPVNTPALRWGC

SEQ ID NO: 6 - protein, 360 aa, Escherichia coli
MDKIIISPAKYIQGNGSLDNIATYAASLGTEPLIIADEFVTGLVGDRVSQSFARENIIADFDVFCGECSQNEISRIRKKFNQRKYNVVIGIGGGKTLDTAKAVAYYQKIPVVVVPTIASTDAPTSSLAVIYTPDGQFSEYLFFPKNPDMVIMDTGVISAAPVRLLVAGMGDALSTWFEARANQASGKATMAGGASTLAALAIARLCYTTLLEDGYKAKVAVEQGVSTKAVENIIEANTYLSGIGFESSGLAAAHAIHNGMTQLEECHHCYHGEKVAFGVLVQLVLENAPQEEIETVLNFCHSVGLPTNLHMLGVKEINKDKLRAVAKAATAEGETIHNMPFVVTAQDVLCAILTAHHLGL

SEQ ID NO: 7 - protein, 422 aa, Escherichia coli
MTSNWKRWITLALCGIAGSAIYKLPYLRETYYDAMQQATGATNAELGFLMTAYGLVNFLLYLPGGWAADRFSARKLMTFSLISTGISGFYYATFPSYTMICLLHALWAVTTVFTFWAVCVRIIRTLGTSEEQGRLYGYWFLGKGLTSIVLGFLSVPVFAKFGEGVDGLRATIIFYSVVTILAGVLAWFVCQDETHSEDKANFRLADMAFVLKMPTVWLAGVVTFCMWSIYIGFGMVTPYLTQILHMGESEVAVASILRAYVLFAMGGLIGGQLADRCASRTRFMIYAFIGMIVFTTVYFFLPGESRYVTIALANMVALGVFIYSANAVFFSIIDEVRIPAKVTGTAAGLISLLTYFPEIYCYTMVGNMVDRKPGIAGYQDVFLFMLVCAFIGLIAALVLQRVNRKSKNQVNEITQNNADCAS

SEQ ID NO: 8 - protein, 454 aa, Escherichia coli
MVDMINESARQTPVIAQTDVLVIGGGPAGLSAAIAAGRLGARTMIVERYGSLGGVLTQVGVESFAWYRHPGTEDCEGICREYEGRARALGFTRPEPQSISEVIDTEGFKVVADQMITESGVEPLYHSWVVDVIKDGDTLCGVIVENKSGRGAILAKRIVDCTGDADIAARAGAPWTKRSKDQLMGVTVMFSCAGVDVARFNRFVAEELKPTYADWGKNWTIQTTGKEDPMFSPYMEDIFTRAQQDGVIPGDAQAIAGTWSTFSESGEAFQMNMVYAFGFDCTDVFDLTKAEIAGRQQALWAIDALRHYVPGFENVRLRNFGATLGTRESRLIEGEIRIADDYVLNQGRCSDSVGIFPEFIDGSGYLILPTTGRFFQIPYGCLVPQKVENLLVAGRCISAGVVAHTSMRNMMCCAVTGEAAGTAAVVSLQQNCTVRQVAIPDLQNTLQQQGVRLA

SEQ ID NO: 9 - protein, 443 aa, Escherichia coli
MHYLKKRFSLIELNVFIPAILFIAVIILCLTIYPQDTSRYINKIHHFLTWEMGGIFLVMTFLVVLCCLWLAFSRYGDILLGQSGEKPDFSLLTWLGLIFTSGTGGSLLYLASVEWIWIIQQPPFGATAGSAQAARWASVYGMFHWGPSAWAWYLICAVPIGWFMHVKKTNSLKVSDLCRGCLGARADGFCGHCVNFFYMFGLLGGAVTSLALGTPMISAVFCHVFHLDPAGQFINVMVIFIWTLVPLFILFFGLKKGVAWASNWNIRADILMLLAILICGPTAFILNQSIDGLGLMLQNFVAMSLSTDAIGRSGFPQMWTVFYFSWWVVYAIPFGLFIARISKGRTIRQLIVCGTLAGSLGCMVFYMVLANFGLSLQTTHVIDFVPILNEQGRGVVVSRLLEQLPASQVFLVAFGAIALISYITGHCTVGYALGFATQKRPDK

SEQ ID NO: 10 - protein, 380 aa, Escherichia coli
MKIVISPDSFKECLPAWKVAEALATGWRKVLPGSQLVCLPVADGGEGTLETLIHATDGTFYTKKVTGPLGESIHAQYGILGNQTTAVIELAQASGLELVSPVQRSPLYTTSFGTGELILAALEHNIDTVILCLGGSATNDGGIGLMSALGASFTDAEGLSVSVNGMGLAAIHHIDLQHLDPRLKNVKFIAACDVTNPLTGDNGATRVFAQQKGASANDLEQLEQGMKNYARCIYRCCGKDVDTIPGSGAAGGVGAALMAFLDARLQPGISLVLEAIQYTQHLKYAALAIVGEGKLDSQSLNGKAPVGAAKVAQMMGVPVIAIAGYIDDQLDLNELRQCGIEACFSVVNGPCNLPTALSQGENNLIRLGENLAGYFRAILS

SEQ ID NO: 11 - protein, 295 aa, Escherichia coli
MNIGFIGTGIMGKPMAYNLQQAGHTLYFSAHFEPAPQEFIGERGIVCSTPREVAQECEVIITMLPDTPHVEDVLFHPNYGVIHGLSHGKIVIDMSSISPVATKAFAQRIIAVGAEYIDAPVSGGEVGAKAGTLSIMVGGCEEVYLQIKPILELMGKNITLVGNVGDGQTCKVANQIIVALNIEAVAEALLFASKSGADPARVREALMGGLASSRVLEVHGERMIKGTFEPGFRISLHQKDLNLALENARLLNTPLPNTATTQQLFSACAALGGKEWDHSALIRALETQANFTIRK

SEQ ID NO: 12 - protein, 579 aa, Escherichia coli
MARMRAIEAAVEILKKEGISVAFGVPGAAINPLYAAMKKSGGIDHILARHVEGASHMAEGYTRSQNGNIGVCIGTSGPAGTDMITGLYSASADSIPILCITGQAPVGKLHKEDFQAVDIEAIATPVTKMARTILEAGQLPGIFQKAFWEMRSGRPGPVLLDLPFDVQMTEIEFDIDLYQPLIPWQPKATRAQAVRALEMLNDAEKPVIIAGGGIINAEASELLREFVELTGVPVIQTLRGWGALSDDHPLMIGRMGCQAGHRYGNASYLASDFVFGIGNRWANRHTGAIETYTEGRKFIHVDIEPAQIGRIFAPDLGIVSDAESALTLFIQVARDMKSRGELKDRSRWIAECAERKRTMLRRSDFDCNPIKPQRVYHEMNKVFGPETRYISTIGLAQIAANQFLHVYRPRHWINACQAGPLGWTMPAALGAVKADPSVPVVAISGDYDFQFLIEELAVGAQFNLPYIHILLNNAYLGLIRQSQRAFDIDYCVQLSFENINAPEINGYGVDHKAVVEGLGCKAIRVFASQDIAPALQEAQRLRDEFHVPVVVEIITERVTNIAMGPDINKVTEFEEILDL

SEQ ID NO: 13 - protein, 260 aa, Escherichia coli
MAKFAANLSMLFTEHPFIERFAQASHAGFHGVEYLFPYDFSTDELASQLHQHNLTQVLFNLPAGNWQEGERGIACHPSRAKEFQEGVCRAIEYAQALNCSQVNCLAGKHPGGYSHEQCHETLVENLRYAVDKLASHGIKLVLEAINTKDIPGFFVNNTRQALNIIYDVNHPDFRYQYDIYHMQIMEGNIATTIKNNLNNIEHIQLADNPGRHEPGTGEINYPWLLNYIDQIGYQGWIGCEYVPSTTTTESLRWLKNETQF

SEQ ID NO: 14 - protein, 624 aa, Escherichia coli
MDIIIMNKESYHNDLKNKWKMFVKHGWVATNSTNHVMLRSWQKCLKHCDPRHWNTPVKASGQTLQTIFSRNEEFIRISQRVVEDHFTLAGDDRLAFLIIDPHGWVLSLNAAGDYSSQLRELGIESGMSWAEDGIGTNVYSLCRETNLYTQLEGAEHFSEQLHCYAMSAAPVIDYYGNIHGYIVCIIETTAELVKLKTSYSCATEIANYIYIENEQKLINKVLCQHNAVIECMDDGFICWNSHSLITMVNSQAQTLLNIDKESLIGQNIRKGFVFPPILNEGITQRNKLSQKQIVLECRGEFIELMVTLRPLSDGSFLLFLHPLDKIRKIAQQQISTNANFTFDSLHAASGGMKQVLLIARRAIKSISPILINGEEGVGKLSLAMAIHNESEQRDGPFISVDCQMLSPENILHELLGSDVGPSPSKFELAHNGTLYLDKVEYLSGEVQSVLLKVLKTGLVTRSDSHRLIPVRFRLITCTSSSLREYVQQGAFSRQLYYEVSMNEIEIPPLRKRREDLKQMIDDIIDKYQERTRKKMTITPDANSVLLEYRWPGNISEFKNRMEKVFINCNRLVLGLENIPLDIRQNNSSGDDDIPHLTSLAELEMQAIAHTCRVCEWNLTKAAED

SEQ ID NO: 15 - protein, 456 aa, Escherichia coli
MEFYLEPARNIPVLATTEVLVVGGGPSGIAAAMSAAREGAATMLIERFGCFGGMMTTAGVESIAWWRHENTVESGGLAREIEETAKSMGASSPEPQSNSQAINAERFKLVADAMLEQAGVRRVLHITAVDVIKQGNNLLGVITESKSGRQAILANVIIDCTGDADIAWFAGAPFIKREREELMCMTTVFSCANINKNAFMQNINSTEPKYGDWGADEENKNWSYDVHESCRDMFSPYLGKVFAKGKSAGIIPKDVTLGGSWSTVTEYGDANYLNVVSIPAVDCTDVFDLTRAEIEGRKQAMQAIEALRQFQPGFEQAQLKNFGMTVGTRESRHIIGRVQLTENDICNEGRHADSIGVFPEFIDGNGHLKLPLEANYFQIPYGVMIPQQVENLLVCGRAIDADNFAYATIRNMGCCIVTGEGAGTAAAIAIKNNTTVSQVDIQTVQERLQQNGVKVF

SEQ ID NO: 16 - protein, 467 aa, Escherichia coli
MKEKPSFYVALTPIIFMMIVLVVGIGVMGWPASVCLLISAAFSCIIAMAKLKYTWDEIQGFIIDKISAVMAPVLIVIFVGFMVATWSYAGTLPMLVYYGMLLVAPAWLYAIAFFLNAVLSYVSGASWGSVASIGVALMGIGSGLHADLPILAAAIVTGAYFGDKLSPLSDTTNLTSAVTKTKLYDLIKYLLWTTIPSTIISLLFFTWLGMIHTSTGSISPESMQMLVQLKELYKFGLLPLLPMLIVLCFTFLRLPTVPALLSSAFVAVLVGWLYQGFPLAEGIKATMSGFRLTMITDSTTLLPSVKELLEQGGLNNQGGFLSFIICAMMFAGILTGTGMMDVTLQSAANKIKSAFGAILASGVLAVIINLLTGSDGLNKIVISELMMKKFEDLNLSPLVLARTLEDFGTMSAPIIPWSAAGLYMATTLGVPTFSYLPYCVFCFCSMIFALIYASTGFRLLRFNEAKA

(19) United States
(12) Patent Application Publication
(10) Pub. No.: US 2005/0064447 A1
(43) Pub. Date: Mar. 24, 2005

(54) Probiotic therapy of neonatal meningitis and method of using E. coli virulence determinatns

(76) Inventor: Sheng-He Huang, Arcadia, CA (US)

Correspondence Address: Raymond Y. Chan, Suite 128, 108 N. Ynez Ave., Monterey Park, CA, 91754, US

(21) Appl. No.: 10/779,304

(22) Filed: Feb. 13, 2004

Related U.S. Application Data
(63) Continuation-in-part of application No. 10/123,965, filed on Apr. 16, 2002.
(60) Provisional application No. 60/284,762, filed on Apr. 18, 2001.

(51) Int. Cl. C12Q 1/68; A61K 39/02

(52) U.S. Cl. 435/6; 424/200.1; 424/93.4

(57) ABSTRACT

Genes and proteins which modulate the virulence of meningitic E. coli bacteria, in particular neonatal meningitic strains of E. coli are provided. In particular, this inventions provides sequences for the IbeA gene cluster (GimA) and the proteins encoded by these genes. Compositions and diagnostic assays utilizing the sequences are also provided.

### CROSS REFERENCE OF RELATED APPLICATION

This is a Continuation-In-Part application of a non-provisional application, application NO. 10/123,965, filed Apr. 16, 2002, which is a regular application of a provisional application with application Ser. No. 60/284,762 filed Apr. 18, 2001.

### STATEMENT REGARDING FEDERALLY SPONSORED RESEARCH

Some of the work described in this application was supported by grant number R29AI40635 from the National Institutes of Health.

### BACKGROUND OF THE INVENTION

1. Field of Invention

The present invention is related to the fields of infectious disease and molecular biology.

2. Description of Related Arts

Escherichia coli (E. coli) is the most common gram negative bacteria causing neonatal meningitis [Huang S H et al., (2000). Microbes and Infection. 2:1137-44; Huang and Jong (2001) Cellular Microbiology 3:277-87]. The pathogenesis of this disease is not completely defined. In the evolutionary process of microbial pathogenesis, gene acquisition is a major event leading to the emergence and evolution of microbial pathogens. Therefore, microbial pathogens causing infectious diseases usually possess traits or sequence signatures that distinguish them from non-pathogenic or commensal strains. The gut microflora including E. coli is acquired rapidly after birth, remains relatively stable throughout the life. These non-pathogenic (probiotic) bacteria are essential for human homeostasis [Huang, S. H. et al.,(2002), Func Integr Genomics 1:331-44]. Strains causing meningitis possess traits that distinguish them from commensal strains of E. coli and other pathogenic strains such as those causing diarrhea and urinary tract infection (UTI). Characteristically, meningitic strains of E. coli are composed of a restricted number of O serogroups (O1, O2, O7, O18, O83), produce S fimbriae, carry the ibeA genetic island (GimA) and are predominately carrying K1 capsule (over 84%). The presence of these features implies that meningitic strains possess a defined set of virulence determinants that allow the bacterium to penetrate the blood-brain barrier and get into the central nervous system. Such clusters of potential virulence genes (based on the sequence information), termed genetic islands, have been suggested for meningitic pathogens N. Meningitidis, E. coli K-1 and H. influenzae but have yet to be defined by in vitro or in vivo experimental approaches (Bloch, C. A. et al., (1996) FEMS Microbiol. Lett.,144: 171-176; Bonacorsi S P et al., (2000) Infect Immun. 68:2096-2101; Chang C C et al.,(2000) Infect Immun 68: 2630-2637; Klee S R et al., (2000) Infect Immun. 68:2082-2095; Rode C K, et al., (1999) Infect Immun. 67:230-236)). The genetic islands such as GimA carry virulence facors that may make E. coli shift from the non-pathogenic to meningitic.

Despite advances in antimicrobial chemotherapy, the mortality and morbidity associated with neonatal gram negative bacillary meningitis has remained high. A better understanding of the bacterial genes and proteins that contribute to the pathogenesis of bacterial meningitis (e.g., breaching of the blood brain barrier (BBB) by bacterial pathogens, induction of apoptosis in human endothelial and neural cells) will facilitate the development of novel treatments and prognostic and diagnostic tools for the disease.

### SUMMARY OF THE PRESENT INVENTION

This invention relates in general to genes and proteins which modulate the virulence of E. coli bacteria, in particular meningitic strains of E. coli. More specifically, the invention relates to the characterization of the genes in the ibeA gene cluster (GimA) and the proteins encoded by these genes, in particular the ibeA gene, to compositions comprising the same and applications utilizing the compositions.

It is an object of this invention to provide isolated or purified nucleic acid sequences encoding the genes of the ibeA gene cluster.

It is another object of this invention to provide amino acids for the proteins encoded by the genes of the ibeA gene cluster.

It is another object of this invention to provide a recombinant molecule comprising a vector and all or part of one or more of the nucleic acid sequences of the ibeA gene cluster.

It is another object of this invention to produce recombinant proteins encoded by the ibeA gene cluster.

It is another object of this invention to provide methods of diagnosing E. coli meningitis.

It is another object of this invention to enhance our understanding of probiotics and their use in the management of infectious diseases, with emphasis on neonatal bacterial meningitis.

It is also an object of this invention to provide vaccines and antimicrobial agents that specifically attack meningitic bacteria but protect probiotic microbes.

These and other objectives, features, and advantages of the present invention will become apparent from the following detailed description, the accompanying drawings, and the appended claims.

### BRIEF DESCRIPTION OF THE DRAWINGS

FIG. 1 shows the complete nucleotide sequence and deduced amino acid sequence of the gene ibeA from Escherichia coli K1 strain. The calculated molecular weight of the full-length protein is 50 kDa. Bolded nucleotide sequences indicate potential ribosome-binding site (RBS), and -10 and -35 promoter regions. The first 15 N-terminal amino acid residues (italicized) completely match the sequence derived from the N-terminal sequencing of the isolated 50-KDa protein expressed in E. coli BL21 (DE3) carrying the ibeA gene (lane 2 in FIG. 2B). Three putative transmembrane domains are underlined. Bolded amino acid sequences denote the partial ORF of ibeA reported previously [Huang S H, et al, Infect Immun (1995); 63:4470-5]. Arrows indicate the TnphoA insertion site (vertical arrows between nt 506 and nt 507 ) and deletion of ZD1 (right arrow, beginning nt151;left arrow beginning at nt 1455) in ibeA, respectively.

FIG. 2 shows In vitro and in vivo biosynthesis of the IbeA protein. Panel A, the products of two in vitro coupled transcriptions/translations in Escherichia coli T7 S30 extract assay system were run in parallel on the same SDS-PAGE (10% polyacrylamide) gel. The sizes of molecular weight markers were indicated to the right. The following templates were added to the reaction mixtures: lane 1, pFN23A carrying a 2.3-kb ibeA locus; lane 2, pFN476 (vector). A 50-Kda protein was produced in pFN23A (lane 1) but not in pFN476 (lane 2). Panel B, SDS-PAGE (10% polyacrylamide) of total protein extracts of IPTG-induced Escherichia coli BL21(DE3) transformed with the vector pFN476 (lane 1), the pFN476-derived plasmid pFN23A bearing the complete ORF of ibeA gene (lane 2). A mixture of protein standards (low molecular markers) was run in lane 3, and their molecular mass values (in kDa) are indicated. Additional bands in lane 1 (FIG. 2A) and lane 2 (FIG. 2B) may result from truncated proteins due to over-expression of IbeA protein.

FIG. 3 shows expression and purification of IbeA proteins. Panel A, SDS-PAGE (10%polyacrylamide) of total protein extracts of IPTG-induced Escherichia coli BL21(DE3)transformed with the vector pET28a(+) (lane 1), the soluble (lane 2) and insoluble proteins (lane3) from the pET28a(+)-derived plasmid pET17A bearing the complete ORF of ibeA gene (1.7 kb). A mixture of protein standards (low molecular markers) was run in lane 4, and their molecular mass values (in kDa) are indicated. The different patterns of protein bands in the soluble (lane 2) and insoluble (lane 3) fractions showed that a 53-kDa protein is predominately present in the inclusion body (lane 3). Panel B, a 53-kDa recombinant IbeA protein witha N-terminal His-tag was expressed in BL21(DE3) carrying pET17A after induction with IPTG. The protein was purified by Ni-NTA Sepharose affinity columns and then refolded by sequential dialysis as described in Materials and Methods. The proteins were resolved on a SDS-polyacrylamide gel, followed by staining with Coomassie Brilliant Blue. Lane 1: purified and refolded IbeA; and lane 2: the molecular weight markers.

FIG. 4 shows inhibition of Escherichia coli K1 invasion of BMEC by affinity purified and refolded IbeA protein. Confluent monolayers of human BMEC were incubated with either BSA (control)(μg/well), or IbeA protein (μg/well) for 1 h at 37° C. before addition of bacteria. Invasion assays were carried out as described in the Materials and Methods in the Examoles. Each value represents the mean of at least four experiments done in triplicate, and the error bars indicate the standard deviations.

FIG. 5 shows Complementation of the non-invasive mutants of E44 with pUC23A and pUC1O30 containing ibeA locus. Escherichia coli E44 is a spontaneous rifampin-resistant mutant of RS218 expressing IbeA. 10A-23 and ZD1 are the TnphoA insertion and isogenic deletion mutants of ibeA derived from E44, respectively. Invasion assays were carried out as described in the Materials and Methods in the Examples. The relative invasion of the mutants compared to the parent strain E44 are shown. Complementation of the TnphoA mutant 10A-23 (A) and the isogenic ibeA deletion mutant ZD1 (B) was presented. Results are means of four separated experiments; bars represent SD.

FIG. 6 shows a schematic representation of the overlapping DNA clones covering the 20.3 kb gimA and DNA sequencing by primer-walking. Three overlapping clones were identified by screening lambdaGEM-12/RS218 genomic DNA library (A10-8 and A10-30) and PCR cloning. The regions containing ibeA common to A10-8 and A10-30, and the overlapping regions between L7 and A10-8 are shown. 30T7 (a-f), 30T7 (1-3), 8T3 (a-c), 8T3 (1-6), 10A5-(2-11), 10A3-(1-7), and L7SP1 represent the primers used for DNA sequencing.

FIG. 7 shows the relative genetic location and operon structures of the genetic island gimA. Open rectangles indicate the ORFs for the GimA and the flanking E. coli K12 genes. The orientation of transcription is indicated by arrows. The GimA consists of 4 operons (GimA1,2,3,4).

FIG. 8 shows the ORF annotation by sequence comparison. 13 of 15 ORFs encoded by the genetic island gimA in E. coli K-1 strain RS218 show significant sequence homology to the corresponding paralogues from E. coli K-12 strain.

FIG. 9 shows the phylogenetic trees of 12 gene products in GimA based on multiple alignments with Clusta1W. ECOK1: E. coli K1strain; ECOK12a: E. coli K12 strain; ECOK12b: E. coli K12 strain; ECOK12c: E. coli K12 strain; CORGL: BACSU: MYCTU: HAEIN: Haemophilus influenzae; SYNY3: AISC: MAVL: MYCL: ALCEU: Ralstonia eutropha; CAEEL: Caenorhabditis elegans; DROME: Drosophila melanogaster; STRCO: Streptomyces coelicolor; TRYBB: VIBPA: PSEPU: BACI: PSEU: CITFR: PICPA: Pichia pastoris; SCHPO: Schizosaccharomyces pombe; PICAN: Pichia angusta; PSEA: ECBK12: GARK12: RHOCA: Rhodobacter capsulatus; SALTY: LACSK: LISMO: STAAU: MYCPN: MCYGE: LYCE: XANCP: Xanthomonas campestris pv. campestris

FIG. 10 shows multiple alignment of 4 sequences of Na(+)/H(+) antiporters, as obtained from CLUSTAL W and DIALIGN. Identical and similar residues in more than 52% of the sequences are drawn on black and shaded backgrounds, respectively. From top row to bottom row, the sequences are IbgT from E. coli K-1 (ECOK1), Na(+)/H+antiporter from H. influenzae (HAEIN)(Q57007), B. firmus (BACFI)(P27611) and B. subtilis (BACSU)(P54571).

FIGS. 11A-11D show the nucleotide sequence for the ibe A gene cluster. The start sites for each gene in the cluster is provided in column 4 of Table 4. For example the start site for PptE is 1694 on the complimentary strand. The proteins encoded by the gimA1 and gimA3 operons are encoded by a nucleotide sequence complimentary to the one shown in FIG. 11A-D.

FIG. 12 shows the amino acid sequences for Pgdk (GimA1); PptE (GimA1); PmpT (GimA1); PdaK (GimA1); CgrD (GimA2); CgxT (GimA2); CdlD (GimA2); Cnit (GimA2 ); GcxK (GimA2 ); GcxR (GimA3); Gc1A (GimA3); GhyI (GimA3); IbgR (GimA4); IbgT (GimA4).

FIG. 13 shows induction of apoptosis by the IbeA protein (upper right panel) and inhibition of induction of apoptosis by the IbeA protein by the bacterial permeability-increasing protein (BPI) (lower right panel; ratio of IbeA:BPI was 1;1). The upper left panel shows incubation of the endothelial cells with BSA and IbeB proteins. the lower left panel shows incubation of the endothelial cells with BPI alone.

### DETAILED DESCRIPTION OF THE INVENTION

The term “nucleotide sequence” refers to, deoxyribonucleic acid (DNA), ribonucleic acid (RNA), and also cDNA.

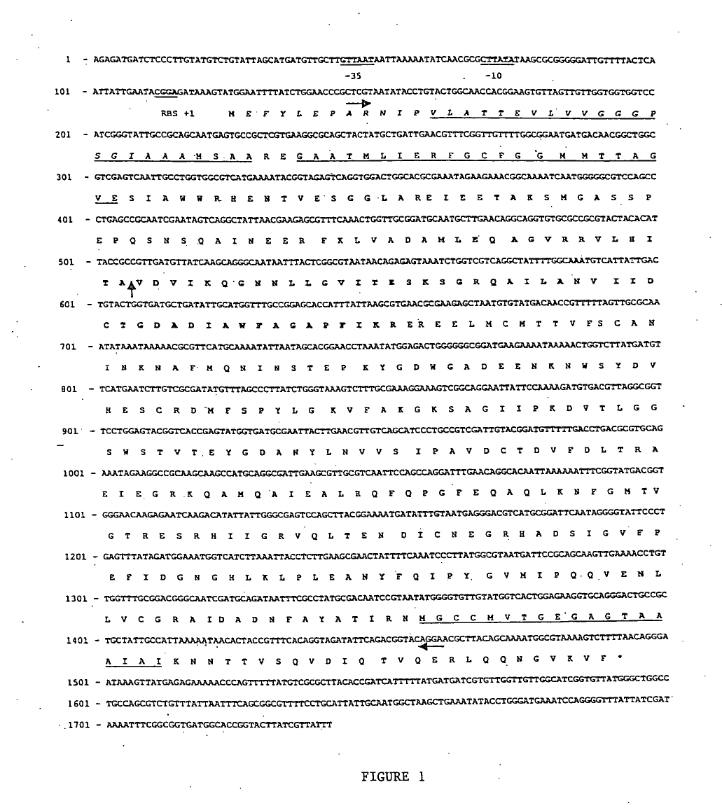

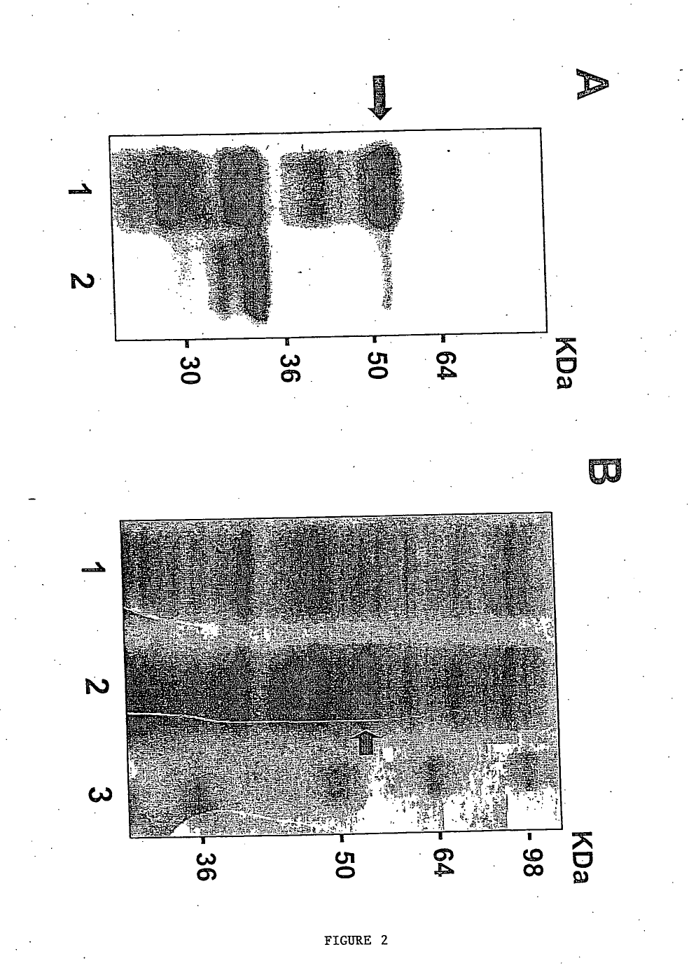

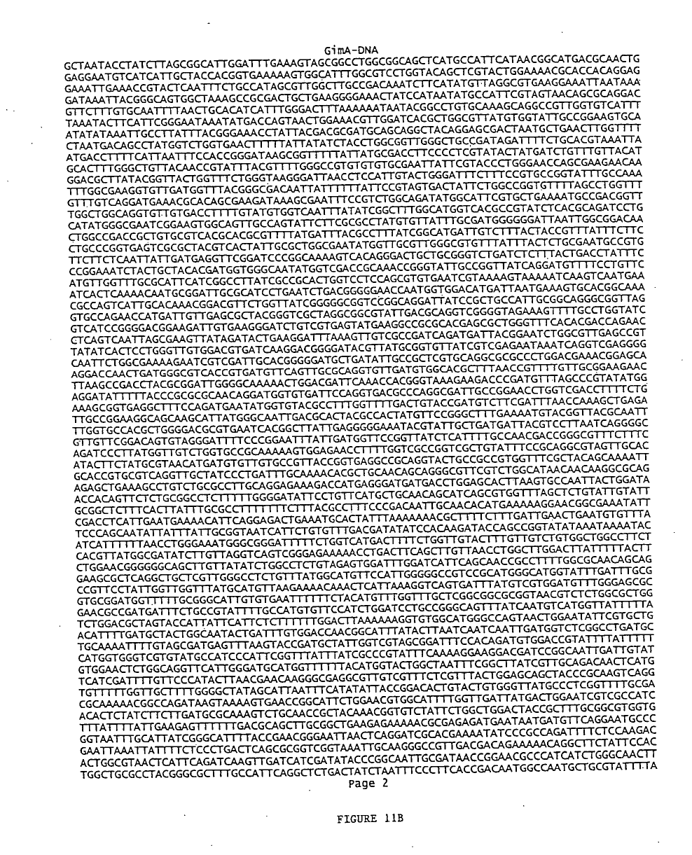

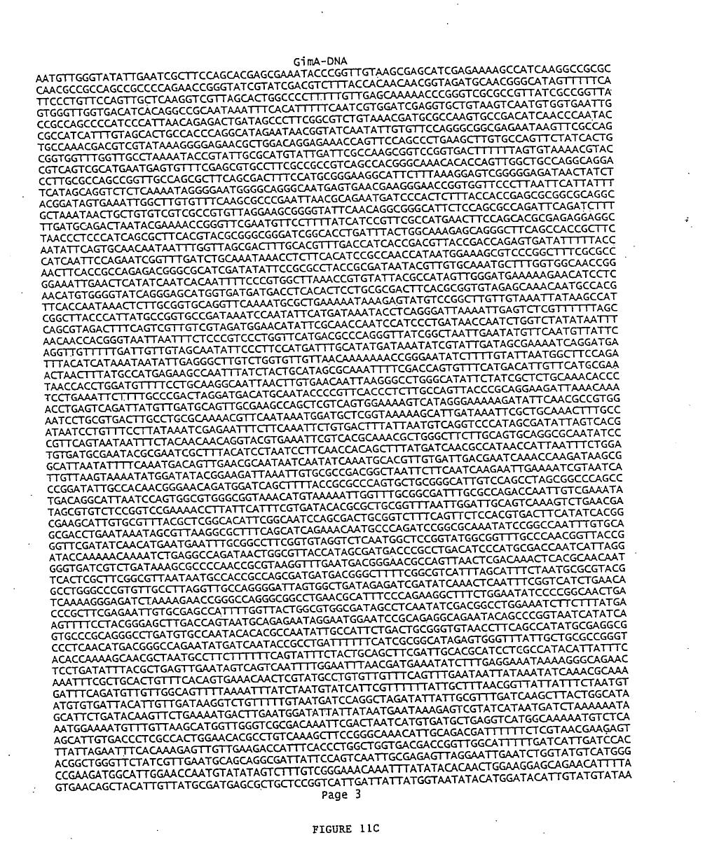

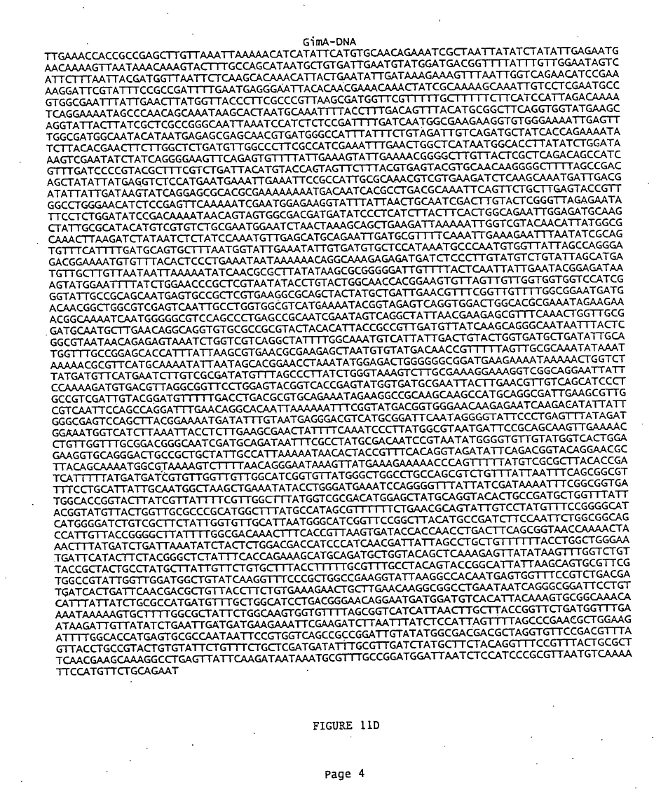

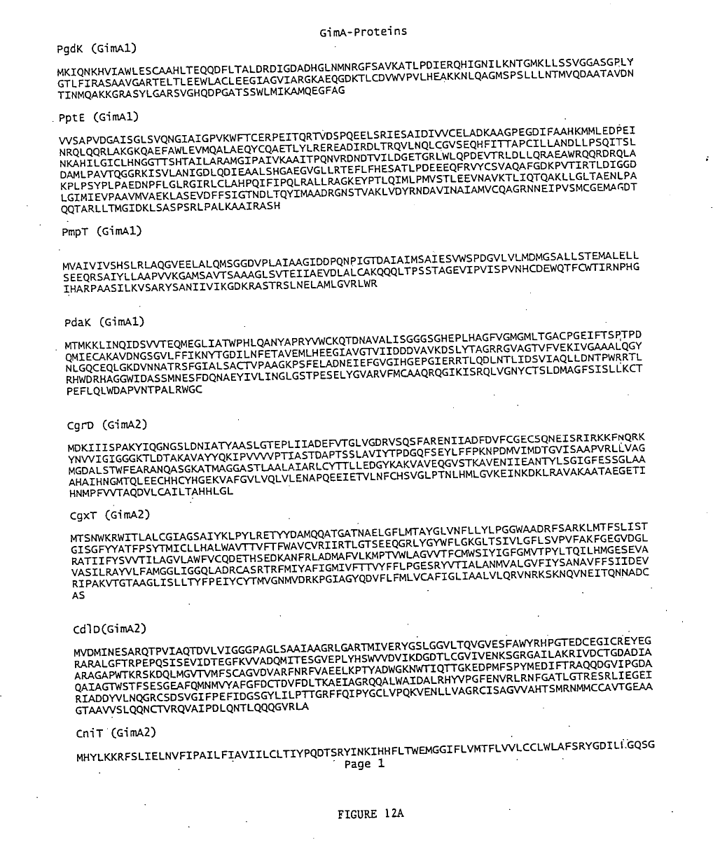

Substantially homologous as used herein refers to substantial correspondence between a nucleic acid gene sequence of the IbeA gene cluster(FIG. 1; FIG. 11). Substantially homologous means about 30-100% homology, preferably by about 60- to 67-100% homology, and most preferably about 80-100% homology between an IbeA gene cluster sequence and that of any other nucleic acid sequence. In addition, substantially homologous as used herein also refers to substantial correspondence between an amino acid sequence of the IBEA gene cluster (shown in FIGS. 1 and 11) and that of any other amino acid sequence.

The term “modulation” refers to either an increase or a decrease in the expression of a gene transcript or protein or impairment of the activity of the protein.

The term “specifically hybridizable” is used to indicate a sufficient degree of complementarity or precise pairing such that stable and specific binding occurs between the a nucleic acid sequence and the DNA or RNA target. It is understood in the art that the sequence need not be 100% complementary to that of its target nucleic acid to be specifically hybridizable.

The term “corresponds to” refers to homologous to or substantially equivalent to or functionally equivalent to the designated sequence.

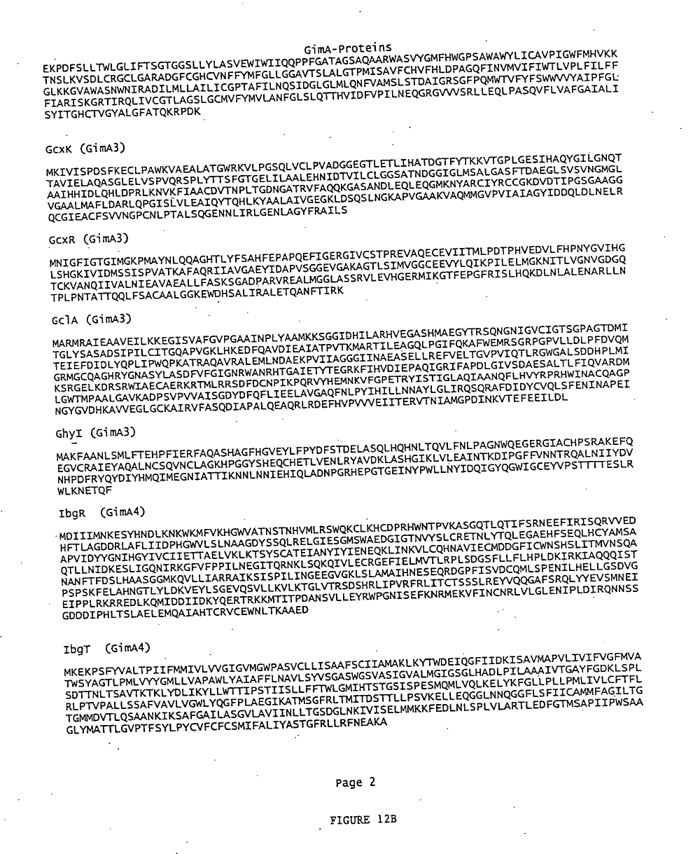

The present invention provides nucleic acid sequences for the genes of the ibeA gene cluster (FIG. 1; FIG. 11) which encode 15 proteins (FIG. 2; FIG. 12) which modulates the virulence of meningitic E. Coli and thus the pathogenicity of a meningitic E. Coli, such as in neonatal bacterial meninginitis. For example, the IbeA gene modulates the ability of the meningitic E. Coli to penetrate the BBB and the induction of apoptosis. The nucleic acid sequences for the IbeA gene cluster shown in FIGS. 1 and 11 (Table 4), represent preferred embodiments of the invention. It is, however, understood by one skilled in the art that due to the degeneracy of the genetic code variations in the gene sequences shown in FIGS. 1 and 11 will still result in a DNA sequence capable of encoding the IBEA protein corresponding to that gene sequence. Such DNA sequences are therefore functionally equivalent to the sequences set forth in FIGS. 1 and 11 and are intended to be encompassed within the present invention. Further, a person of skill in the art will understand that there are naturally occurring allelic variations in a given species of the nucleic acid sequences shown in FIGS. 1 and 11, these variations are also intended to be encompassed by the present invention.

This invention further includes proteins or polypeptide or analogs thereof having substantially the same function as any one of the proteins of the IbeA operon or gene cluster proteins of this invention. Such proteins or polypeptides include, but are not limited to, a fragment of the protein, or a substitution, addition or deletion mutant of a protein. This invention also encompasses proteins or peptides that are substantially homologous to any one of the proteins produced by the IbeA gene cluster. Substantially homologous means about 70-100% homology, preferably about 80-100% homology, and most preferably about 90-100% homology between any one of the proteins of the invention and any another amino acid sequence or protein or peptide.

The term “analog” includes any polypeptide having an amino acid residue sequence substantially identical to any one of the amino acid sequences specifically shown herein (FIGS. 1 and 12) in which one or more residues have been conservatively substituted with a functionally similar residue and which displays the functional aspects of any one of the proteins described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid or another.

The phrase “conservative substitution” also includes the use of a chemically derivatized residue in place of a non-derivatized residue. “Chemical derivative” refers to a subject polypeptide having one or more residues chemically derivatized by reaction of a functional side group. Examples of such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those proteins or peptides which contain one or more naturally-occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. Proteins or polypeptides of the present invention also include any protein or polypeptide having one or more additions and/or deletions or residues relative to a sequence encoded by a nucleotide sequence of the IbeA gene cluster.

This invention also provides a recombinant molecule comprising all or part of one or more of the nucleotide sequences (FIGS. 1 and 11) and a vector. Expression vectors and method of producing expression vectors are well known in the art Generally, expression vectors suitable for use in the present invention comprise a least one expression control element operationally linked to the nucleic acid sequence. The expression control elements are inserted in the vector to control and regulate the expression of the nucleic acid sequence. It will be understood by one skilled in the art the correct combination of required or preferred expression control elements will depend on the host system chosen.

It will further be understood that the expression vector should contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods (Ausubel et al., (1987) in “Current Protocols in Molecular Biology”, John Wiley and Sons, New York, N.Y.) or commercially available.

Another aspect of this invention relates to a host organism or cell into which the recombinant expression vector has been introduced. Examples of host cells that may be used include, but are not limited to, eukaryotes, such as animal (e.g., endothelial cells, epithelial cells), plant, insect and yeast cells and prokaryotes, such as E. coli. The means by which the vector carrying the gene may be introduced into the cell includes, but is not limited to, microinjection, electroporation, transduction, or transfection using DEAE-dextran, lipofection, calcium phosphate or other procedures known to one skilled in the art (Sambrook et al. (1989) in “Molecular Cloning. A Laboratory Manual”, Cold Spring Harbor Press, Plainview, N.Y.). In a preferred embodiment, eukaryotic expression vectors that function in eukaryotic cells are used. Examples of such vectors include, but are not limited to, retroviral vectors, vaccinia virus vectors, adenovirus vectors, herpes virus vector, fowl pox virus vector, bacterial expression vectors, plasmids, or the baculovirus transfer vectors. peferred vectors include, but are not limited to pET28a and pCMV-HA. Preferred eukaryotic cell lines include, but are not limited to, endothelial or epithelial cells.

In a further embodiment, the recombinant protein expressed by the host cells can be obtained as a crude lysate or can be purified by standard protein purification procedures known in the art which may include differential precipitation, molecular sieve chromatography, ion-exchange chromatography, isoelectric focusing, gel electrophoresis, affinity, and immunoaffinity chromatography and the like. (Ausubel et. al., (1987) in “Current Protocols in Molecular Biology” John Wiley and Sons, New York, N.Y). In the case of immunoaffinity chromatography, the recombinant protein may be purified by passage through a column containing a resin which has bound thereto antibodies specific for a protein of the invention (Ausubel et. al., (1987) in “Current Protocols in Molecular Biology” John Wiley and Sons, New York, N.Y.).

The nucleotide sequences or portions thereof, of this invention are useful as probes for the detection of any one of the genes of the IbeA gene cluster or detection of any one of the gene products (e.g., mRNAs) in for example, a biological sample. Isolation of nucleic acids from a biological sample may be performed by standard methodology (Ausubel et al., (1987) in “Current Protocols in Molecular Biology”, John Wiley and Sons, New York, N.Y.). Detection may be performed by a variety of conventional methodologies standard methodology, including, but not limited to, Northern Blot Analysis, PCR etc (Ausubel et al., (1987) in “Current Protocols in Molecular Biology”, John Wiley and Sons, New York, N.Y.). The probes of the present invention are preferably labeled to provide for detection. Examples of labels include, but is not limited to, radioactive labels, fluorescent lables, photometric labels or chemical labels (Ausubel et al., (1987) in “Current Protocols in Molecular Biology”, John Wiley and Sons, New York, N.Y.) In a preferred embodiment a biological sample is assayed for the presence of the IbeA or IbeB genes or gene products. The nucleotide sequences of this invention (FIGS. 1 and 11) can also be used as probes to isolate homologs in other species.

The nucleotide sequences or portions thereof, of this invention are useful in diagnostic assays for meningitic E. coli, in particular neonatal meningitic E. Coli. The diagnostic assays may be performed as described above to detect nucleic acid sequences from a biological sample which are complimentary to the nucleic acid sequences of the invention. in a biological sample. By way of example, the diagnostic assay may comprise an array of the nucleic acids of the invention attached to a support (e.g., dot blots on a nylon hybridization membrane Sambrook et al.,) that is contacted with the nucleic acids isolated from the biological sample nylon. In a preferred embodiment for the diagnostic assay the nucleic acid sequences comprise a microarray.

The nucleic acid sequences of the invention may be utilized as probes in microarrays comprising a solid phase on the surface of which are immobilized a population of the nucleic acids of the invention. Microarrays can be generated in a number of way The probes can be attached to a solid support or surface, which may be made from, for example, glass, plastic (e.g., polypropylene, nylon), polyacrylamide, nitrocellulose, or other materials. Methods for attaching the nucleic acids to the surface of the solid phase include, but are not limited to, printing on glass plates (Schena et al, 1995, Science 270:467-470; DeRisi et al, 1996, Nature Genetics 14:457-460; Shalon et al., 1996, Genome Res. 6:639-645; and Schena et al., 1995, Proc. Natl. Acad. Sci. U.S.A. 93:10539-11286); or ink jet printer; for oligonucleotide synthesis (U.S. application Ser. No. No. 09/008,120, filed Jan. 16, 1998).

The microarrays can also be high-density oligonucleotide arrays. Techniques are known for producing arrays containing thousands of oligonucleotides complementary to defined sequences (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270; Blanchard et al., Biosensors & Bioelectronics 11:687-690). Other methods for making microarrays may also be utilized (Maskos and Southern, 1992, Nuc. Acids. Res. 20:1679-1684; U.S. Pat. No. 6,136,592; WO 200054883; WO 200055363; WO 200053812; WO 200014273). The microarrays may be used as is or incorporated into a biochip, multiwell or other device.

Preferably the microarrays of the present invention comprise, in addition to one or more of the nucleic acids of the present invention, nucleic acids from non-meningitic strains of E. coli as a control. In a preferred embodiment, the entire IbeA gene cluster is included in the microarray.

One of skill in the art will understand that the hybridization and wash conditions are chosen so that the nucleic acid sequences to be analyzed by the invention (e.g., the nucleic acids islolated from the biological sample) “specifically bind” or “specifically hybridize” to the nucleic acid sequences the array. Optimal hybridization conditions will depend on the length (e.g., oligomer versus polynucleotide greater than 200 bases) and type (e.g., RNA, or DNA) of probe and target nucleic acids. General parameters for specific (i.e., stringent) hybridization conditions for nucleic acids are described in Sambrook et al., (supra), and in Ausubel et al., 1987, Current Protocols in Molecular Biology, Greene Publishing and Wiley-Interscience, New York).

Examples of biological samples that can be used in the above assays include, but are not limited to, cerebral spinal fluid, blood, urine, biopsy specimens, pathology specimens, and necropsy specimens. The nucleotide sequences of this invention (FIGS. 1 and 11) can also be used as probes to isolate homologs in other species.

This invention further comprises an antibody or antibodies reactive with the IBEA protein or polypeptides or portion thereof. In this embodiment of the invention the antibodies are monoclonal or polyclonal in origin and are produced by conventional methodology (Kohler and Milstein (1975) Nature 256, 495-497; Campbell “Monoclonal Antibody Technology, the Production and Characterization of Rodent and Human Hybridomas” in Burdon et al. (eds.) (1985) “Laboratory Techniques in Biochemistry and Molecular Biology,” Volume 13, Elsevier Science Publishers, Amsterdam; PCT patent applications: publication number WO 901443, WO 901443 and WO 9014424 and in Huse et al. (1989) Science 246:1275-1281). The protein or portions thereof used to generate the antibodies may be isolated from the meningenitic E. coli strain K1, recombinantly produced, or commercially synthesized (Merrifield, R. B. (1963) J. Amer. Soc. 85:2149). If the portion of the protein selected for generating antibodies is to short to be antigenic it may be conjugated to a carrier molecule to enhance the antigenicity of the peptide. Examples of carrier molecules, include, but are not limited to, human albumin, bovine albumin and keyhole limpet hemo-cyanin (“Basic and Clinical Immunology” (1991) Stites, D. P. and Terr A. I. (eds) Appleton and Lange, Norwalk Conn., San Mateo, Calif.).

The antibodies of this invention may be used in immunoassays to detect the novel proteins in biological samples. Examples of immunoassays that may be used include, but are not limited to, radioimmunoassay, Western blot assay, immunofluorescent assay, enzyme immunoassay, chemiluminescent assay, immunohistochemical assay and the like. (In “Principles and Practice of Immunoassay” (1991) Christopher P. Price and David J. Neoman (eds), Stockton Press, New York, N.Y.; Ausubel et al. (eds) (1987) in “Current Protocols in Molecular Biology” John Wiley and Sons, New York, N.Y.). Biological samples appropriate for such detection assays include, but are not limited to, blood samples, cerebral spinal fluid, urine, biopsy specimens, pathology specimens, and, necropsy specimens. Proteins may be isolated from biological samples by conventional methods described in (Ausubel et al., (eds) (1987) in “Current Protocols in Molecular Biology” John Wiley and Sons, New York, N.Y.).

The proteins or portions thereof of the invention may be used as a vaccine. The vaccine may be provided prior to any clinical indicia of bacterial meningitis or during an infection to enhance the patient's own immune response to the meningitic pathogens carrying virulence proteins but not probiotic bacteria. The vaccine, which acts as an immunogen may comprise one or more of the proteins of the invention or portions thereof. Vaccination can be conducted by conventional methods. For example, the immunogen can be used in a suitable diluent such as saline or water, or complete or incomplete adjuvants. Further, the immunogen may or may not be bound to a carrier to make the protein immunogenic. Examples of such carrier molecules include but are not limited to bovine serum albumin (BSA), keyhole limpet hemocyanin (KLH), tetanus toxoid, and the like. The immunogen also may be coupled with lipoproteins or administered in liposomal form or with adjuvants. The immunogen can be administered by any route appropriate such as intravenous, intraperitoneal, intramuscular, subcutaneous, and the like. The immunogen may be administered once or at periodic intervals until a significant titer of anti-GIMA immune cells or anti-GIMA antibody is produced. Alternatively the expression vectors of the invention may be utilized as vaccines. For example, an expression vector comprising all or part of the gene sequence for the GimA genes including ibeA, whose proteins have been shown to be immunogenic virulence factors can be used. The GimA genes can be cloned into a plasmid vector such as pVR1020 (Vical, Inc., San Diego, Calif.). The vaccine may be tested in an animal model such as mice. This DNA vaccine can be delivered to mice by intradermal inoculation and the antibody titers in the antisera from the immunized mice measured by enzyme-linked immunosorbent assay. The elicited antibodies can also be tested by immunoblotting with GimA proteins including IbeA. Following the initial immunization and a few (e.g., 2-4) consecutive boosts, each at 2-week intervals, protection can be tested in a neonatal mouse model of E. coli meningitis.

The antibodies of the invention can also be administered to a subject as anti-meningitic bacterial agents. Preferably the antibodies administered are designed so to minimize an adverse immune response to the antibody itself (e.g., chimeric antibodies, humanizes antibodies; Robinson et al., International Patent Application 184,187; Taniguchi M., European Patent Application 171,496; Morrison et al., European Patent Application 173,494; Neuberger et al., PCT Application WO 86/01533; Cabilly et al., 1987 Proc. Natl. Acad. Sci. USA 84:3439; Nishimura et al., 1987 Canc. Res. 47:999; Wood et al., 1985 Nature 314:446; Shaw et al., 1988 J. Natl. Cancer Inst. 80: 15553; Morrison S., 1985 Science 229:1202 and by Oi et al., 1986 BioTechniques 4:214.).

While it is possible for the vaccines or antibodies be administered in a pure or substantially pure form, it is preferable to present it as a pharmaceutical composition, formulation or preparation. The formulations of the present invention, are for both veterinary and human use, comprises one or more of the vaccines or antibodies of the present invention together with one or more pharmaceutically acceptable carriers and, optionally, other active agents (e.g., additional antigens for a multivalent vaccine, antibiotics, BPI etc) or therapeutic ingredients. The carrier(s) must be “acceptable” in the sense of being compatible with the other ingredients of the formulation and not deleterious to the recipient thereof. The characteristics of the carrier will depend on the route of administration. Such a composition may additionally contain carrier, diluents, fillers, salts, buffers, stabilizers, solubilizers, and other materials well known in the art. The formulations may be prepared by any method well-known in the pharmaceutical art.

Upon formulation, solutions will be administered in a manner compatible with the dosage formulation and in a effective amount and a variety of dosage forms. For example, an effective concentration of the compositions of the invention may be administered orally, topically, intraocularly, parenterally, intranasally, intravenously, intramuscularly, subcutaneously, transdermally or by any other effective means. These particular aqueous solutions are especially suitable for intravenous, intramuscular, subcutaneous, intraperitoneal, oral, intercranial, cerebrospinal fluid, pleural cavity, occular, or topical (lotion on the skin) administration. Upon formulation, solutions will be administered in a manner compatible with the dosage formulation and in a effective amount and a variety of dosage forms.

This invention also relates to a method of treating bacterial meningitis, in particular neonatal meningitis with probiotics/prebiotics and the bactericidal/permeability-increasing protein (BPI). Probiotics and prebiotics are known to enhance normal microflora by replenishing suppressed nonpathogenic bacteria and inhibiting the growth of microbial pathogens. For example, Lactobacillus casei GG (LGG) has been used in the treatment of a variety of infant and childhood intestinal disorders, including diarrhea, malabsorption, and Clostridium difficile colitis [Mattar, A. F. et al., (2002), Pediatr Surg Int. 18:586-90.]. It is suggested that in the breast-fed infant, elevated gut Bifidobacterium may provide health advantages in comparison with formula-fed infants. Probiotic bacteria such as Lactobacillus, Bifidobacterium and Bacteroides thetaiotaomicron may suppress the growth of meningitic pathogens. Treatment with probiotics may be tested in a neonatal murine model of E. coli meningitis. BPI binds with high affinity to endotoxin and, as demonstrated herein(see Example 13) blocks IbeA-induced apoptosis. Treatment with BPI may first be tested in a neonatal murine model of E. coli meningitis. Dosages for human clinical trials may be based on clinical trials in progress utilizing BPI. One of skill in the art will appreciate that individualization of dosage may be required to achieve the maximum response for a given subject. It is further understood by one skilled in the art that the dosage administered to a individual being treated may vary depending on the individuals age, severity or stage of the infection and response to the course of treatment. One skilled in the art will know the clinical parameters to evaluate to determine proper dosage for the subject being treated by the methods described herein. Such dosages may be administered as often as necessary and for the period of time judged necessary by the physician.

This invention also relates to a screening assay for assessing the therapeutic potential of a candidate agent for inhibiting the apoptotic activity of the IBEA protein. By way of example, the therapeutic potential of a candidate agent may be assessed by the assay described in Example 13. A variety of cells may be used in this assay, including, but not limited to, human cells (e.g., endothelial or epithelial). The candidate agents suitable for assaying in the methods of the subject application may be any type of molecule from, for example, chemical, nutritional or biological sources. The candidate agent may be a naturally occurring or synthetically produced. For example, the candidate agent may encompass numerous chemical classes, though typically they are organic molecule, preferably small organic compounds having a molecular weight of more than 50 and less than about 2,500 Daltons. Such molecules may comprise functional groups necessary for structural interaction with proteins or nucleic acids. By way of example, chemical agents may be novel, untested chemicals, agonists, antagonists, or modifications of known therapeutic agents.

The agents may also be found among biomolecules including, but not limited to, peptides, saccharides, fatty acids, antibodies, steroids, purines, pryimidines, toxins conjugated cytokines, derivatives or structural analogs thereof or a molecule manufactured to mimic the effect of a biological response modifier. Examples of agents from nutritional sources include, but is not limited to, extracts from plant or animal sources or extracts thereof. Agents also include antisense oligonucleotides, including antisense peptide nucleic acids ( Good et al., (2001) Nature Biotechnology 19: 360)

The agents may be obtained from a wide variety of sources including libraries of synthetic or natural compounds. Alternatively, libraries of natural compounds in the form of bacterial, fungal, plant, and animal extracts are available or readily produced, natural or synthetically produced libraries or compounds are readily modified through conventional chemical, physical and biochemical means, and may be used to produce combinatorial libraries. Known pharmacological agents may be subjected to random or directed chemical modifications, such as acylation, alkylation, esterification, amidification, etc. to produce structural analogs.

Also provided are kits for performing the assays of the invention (e.g., the diagnostic assay). Such kits may comprise the microarrays of the invention. The microarrays may be incorporated into a biochip or multiwell configuration or any other configuration. The kits may further comprise one or more a, additional regents for performing the assay such as for example buffers, primers, enzymes, labels and the like. The kits may further comprise, or be packaged with, an instrument for assisting with the performance or analysis of the assay. Such an instrument may be an inhalant, syringe, pipette, forceps, measured spoon, eye dropper or any such medically approved delivery vehicle. The kits of the invention may also include an instruction sheet defining administration of the antisense oligonucleotides. The kits of the present invention also will typically include a means for containing the vials in close confinement for commercial sale such as, e.g., injection or blow-molded plastic containers into which the desired vials are retained. Other instrumentation includes devices that permit the reading or monitoring of reactions.

All books, articles, and patents referenced herein are incorporated by reference. The following examples illustrate various aspects of the invention and in no way intended to limit the scope thereof.

### EXAMPLES

### Example 1-6

In order to identify Escherichia coli structures that contribute to the invasion of BMEC, we have previously used transposon TnphoA mutagenesis to generate a collection of noninvasive mutants. Four noninvasive mutants, 10A-23, 7A-33, 23A-20, and 27A-6 with a single TnphoA insertion in ibeA, ibeB, yijP, and aslA , respectively, were found to be significantly less invasive in BMEC monolayer in vitro and in the newborn rat model of hematogenous Escherichia coli meningitis (Hoffman J A, et al, Infect Immun (2000) 68:5062-7; Huang S H, et al, Infect Immun (1995); 63:4470-5; Huang S H, et al, Infect Immun (1999); 67:2103-9; Wang Y, et al, Infect Immun (1999); 67:4751-6).

The partial internal sequence of ibeA (ibe10) gene encoding an 8.2-kDa protein region has been cloned by PCR [Huang S H, et al, Infect Immun (1995); 63:4470-5] and its recombinant protein was able to inhibit Escherichia coli K1 invasion of BMEC monolayers. The ibeA gene has been found commonly in cerebrospinal fluid (CSF) isolates of Escherichia coli K1 (e.g., C5 and RS218), whereas laboratory strains of Escherichia coli K12 (e.g., DH5α and HB101) as well as noninvasive Escherichia coli K1 (e.g., E412) lack ibeA [Huang S H, et al, Infect Immun (1995); 63:4470-5; Bingen E, et al, J Infect Dis (1998); 177:642-50; Bonacorsi S P, et al, Infect Immun (2000); 68:2096-101]. In addition, we have identified an approximately 55-kDa receptor protein (IbeAR) on the surface of BMEC that interacts with Escherichia coli invasion protein IbeA by using IbeA-Ni-Sepharose affinity chromatography [Prasadarao N V, et al, Infect Immun (1999); 67:1131-8], suggesting that IbeA contributes to Escherichia coli K1 invasion of BMEC via a ligand-receptor interaction.

To assess the importance of IbeA-mediated invasion in the crossing of the blood-brain barrier, the biological activity of the invasion protein encoded by the full-length gene and the isogenic deletion mutant of ibeA should be tested. In this work, we further characterized ibeA gene and its invasion protein by chromosomal gene replacement, complementation, in vitro translation, and in vivo protein expression.

### Materials and Methods for Example 1-6

Bacterial strains, plasmids and media. Bacterial strain, plasmid vectors, and their relevant characteristics are described in Table 1. Mutant strains used in this study were derived from E44,a spontaneous rifampin-resistant mutant of a CSF isolate of K1 encapsulated Escherichia coli RS218 (O18:K1:H7), which has been characterized [Kim, K S, et al, J Clin Invest (1992); 90:897-905. Huang S H, et al, Infect Immun (1995); 63:4470-5. Silver R P, et al, Infect Immun (1980); 29:200-6]. DH5α was used as the host strain in subcloning and preparation of plasmids for DNA sequence determination. BL21(DE3) carrying T7 RNA polymerase gene was the host strain for protein expression IbeA. SM10(λpir) and DH5α(λpir) were utilized for making isogenic deletion mutants of ibeA [Donnenberg M S, et al, Infect Immun (1990); 58:1565-71. Donnenberg M S, et al, Infect Immun. (1991); 59:4310-7]. Strains containing plasmids were grown at 37° C. in L broth (10 g of tryptone, 5 g of NaCl, and 5 g of yeast extract per liter) with ampicillin (100 μg/ml), kanamycin (50 μg/ml) and rifampin (100 μg/ml) for positive selection of plasmids or bacterial strains (see Table 1). Bacteria were cultured in L broth and stored in L broth plus 20% glycerol at −70° C.

Chemicals and enzymes: Restriction endonucleases, T4 DNA ligase, and other enzymes were purchased from New England Biolabs (Beverly, Mass.) unless otherwise noted. Chemicals were purchased from Sigma (St. Louis, Mo.). All isotopes were obtained from New England Nuclear Corp. (Boston, Mass.). Reagents for preparation of DNA sequencing gels were ultra pure quality from National Diagnostics (Atlanta, Ga.). The reagents for DNA sequencing reaction with Sequenase and other chemicals were purchased from United States Biochemical Corp. (Cleveland, Ohio). DNA sequencing kits with dye terminators were obtained from PE Applied Biosystem (Foster City, Calif.).

Extraction and manipulation of plasmids and subcloning. All genetic manipulations were performed by standard methods [Sambrook J, Fritsch E F, Maniatis T. Molecular cloning: a laboratory manual, 2nd ed. Cold Spring Harbor, New York; Cold Spring Harbor Laboratory Press, 1989:1.1-18.57]. Plasmid DNA was extracted by using a Plasmid Mini kit (Qiagen Inc., Chatsworth, Calif.). Purification of DNA fragments and extraction from agarose gel slices were performed with Geneclean (Bio 101, La Jolla, Calif.). Competent cells of Escherichia coli were made in 10% glycerol and transformed with electroporation as previously described [Huang S H, et al, Infect Immun (1995); 63:4470-5; Huang S H, et al, Infect Immun (1999); 67:2103-9].

Invasion assays: Invasion assays were performed in human BMECs as previously described [Huang S H, et al, Infect Immun (1995); 63:4470-5. Huang S H, et al, Infect Immun (1999); 67:2103-9. Kim K S, et al, Subcell Biochem (2000); 33:47-59. Stins M F, et al, Am J Pathol. (1994); 145:1228-36. Stins M F, et al, J Neuroimmunol (1997); 76:81-90]. Approximately 10⁷ bacteria were added to confluent monolayers of BMECs with a multiplicity of infection of 100. A percent invasion was calculated by [100×(number of intracellular bacteria recovered)/(number of bacteria inoculated)]. The results were expressed as relative invasion (percent invasion as compared to the invasion of the parent Escherichia coli K1 strain ).

Construction and screening of a genomic library of Escherichia coli RS218. High molecular weight chromosomal DNA was purified from Escherichia coli K1 strain RS218 as previously described [Huang S H, et al, Infect Immun (1995); 63:4470-5]. Genomic DNA was partially digested with Sau3AI (New England Biolabs, Beverly, Mass.) (15 to 23 kb) and then partially filled-in with dGTP and dATP. This DNA with a 5′ protruding overhang (5′-GA-3′) was ligated into LambdaGEM™-12 arms with a compatible XhoI Half-site (5′-TC-3′). Ligation and packaging of recombinant lambda phage were performed according to the manufacturers instructions (Promega, Madison, Wis.). The Escherichia coli genomic library was screened by DNA hybridization [Huang S H, et al, Infect Immun (1995); 63:4470-5] to identify phage clones that contained ibeA. A 0.58-kb ibeA DNA fragment in pCIB10B [Huang S H, et al, Infect Immun (1995); 63:4470-5] was released with EcoRI and purified by preparative agarose electrophoresis and Geneclean (Bio101, La Jolla, Calif.), labeled with [α³²P]dCTP by using an oligolabeling kit (Pharmacia, Peapack, N.J.), and used as a probe in the screening (>1×10⁸ cpm/μg). The phage plaques were replicated onto Nylon filters, UV-linked and hybridized as described previously [Huang S H, et al, Infect Immun (1995); 63:4470-5. Huang S H, et al, DNA Cell Biol (1994); 13:461-71]. Plaques hybridizing to the probe were identified by autoradiography and then purified.

In Vitro Transcription and Translation. In order to determine the size of ibeA ORF, the DNA fragments containing ibeA gene were transcribed and translated in vitro using an Escherichia coli T7 S30 extract assay system according to the manufacturer's instructions (Promega, Madison, Wis.). The reactions were carried out in 50 μl of reaction mix supplemented with 20 μCi of [³⁵S]methionine and 2 μg of purified pFN476 or recombinant pFN23A at 30° C. for up to 2 hr. 5 μl of each reaction containing ³⁵S-labeled proteins was resolved by 10% SDS-PAGE, and gels were dried and then exposed to Kodak X-Omat film overnight.

Protein expression in vivo and N-terminal sequencing. A 2.3 kb SphI DNA fragment carrying ibeA was cloned and expressed in a low-copy-number plasmid vector, pFN476, which has been successfully used to perform expression analysis of a dozen cloned chromosomal genes of Escherichia coli K12 for producing a definitive expression map [21]. The recombinant plasmid pFN23A and its vector (pFN476) were used to transform an Escherichia coli strain, BL21(DE3) which includes an integrated T7 RNA polymerase gene. Escherichia coli genes in this vector can be preferentially expressed in BL21 (DE3) by utilizing its T7 promoter [Studier F W, et al, Methods Enzymol (1990); 185:60-89]. In order to eliminate gene expression from chromosome, rifampin was used to shut down Escherichia coli polymerase before addition of IPTG. Total proteins were subjected to SDS-10% polyacrylamide gel electrophoresis. After separation, the proteins were transferred onto a PVDF membrane. The band corresponding to a 50 kDa protein over-expressed in the transformants with pFN23A was excised and subjected to N-terminal amino acid sequencing.

DNA sequencing and analysis: The complete nucleotide sequence of ibeA was determined by the dideoxy chain termination method of Sanger et al [Sanger F, et al, Proc Natl Acad Sci USA (1977); 74:5463-7] with Sequenase version 2.0 kit from U.S. Biochemicals Corp. (Cleveland, Ohio) and [³⁵S]dATP(1,000 to 1,500 Ci/mmol) obtained from Du Pont NEN Research Products (Boston, Mass.). The flanking DNA sequence of the partial ibeA gene was determined with primer walking. Both strands of the DNA were re-sequenced by the automated approach with fluorescence labeled nucleotides (373A ABI Automated Sequencer) to ensure the accuracy and the sequence data was analyzed with the DNA analysis program developed by the Genetics Computer Group of the University of Wisconsin. DNA and deduced protein sequences were used to search the DNA and protein databases at the National Center for Biotechnology Information (National Library of medicine, Washington, D.C.) by using the BLAST algorithm.

Construction of isogenic in-frame deletion mutant. In order to determine the role of ibeA gene in the pathogenesis of Escherichia coli meningitis, the ibeA in-frame deletion mutant was generated by integration of the recombinant suicide plasmid pVZD. pVZD was constructed as follow. Two PCR DNA fragments, Z (0.9-kb) and D (1.2-kb), flanking a 1.3 kb region to be deleted were generated by using two pairs of primers (10A5-4a/10ZM6 for Z and 10D5M/10D3 for D) and then ligated to make a 2.1 kb fragment (ZD) carrying an ibeA internal deletion (Table 2). The ZD fragment was subcloned into pCVD442 [17] with SmaI. The mutants were obtained by mating E44 with SM10λpir carrying pVZD and selected on LB agar containing ampicillin and rifampin. A single such colony was picked and grown to the late logarithmic phase in LB broth without selection. The dilutions were plated on LB plates containing no NaCl and 5% sucrose. Sucrose-resistant colonies were tested for loss of ampicillin-resistance, indicative of the loss of vector sequence. PCR and DNA sequencing were used to confirm the internal deletion in pVZD and the desired chromosomal gene ibeA of the mutant with primers 10A5-3Sa and 10B3-4a (Table 2). Amplification was carried out by using the following cycle profile: 35 cycles: 94° C. for 1 min, 58° C. for 1 min and 70° C. for 1.5 min [Kolmodin L A, et al, Methods Mol Biol (1997); 67:1-15].

Complementation analysis. A 2.3-kb SphI fragment containing the complete ibeA open reading frame was released from pUC1030 containing an 18 Kb DNA fragment with SphI and subcloned into pUC13. The construct was designated pUC23A. Strain E44 and the mutant ZD1 were transformed with the vector pUC13, and the recombinant plasmids pUC23A and pUC1030. E44 was transformed with pUC13. The transformants were tested for their ability to invade BMEC.

Constructs for making a recombinant IbeA protein with a N-terminal His-tag. The following constructs were made as described previously [Huang S H, et al, Infect Immun (1995); 63:4470-5]. The BamHI-EcoRI fragment (1.7 kb) from pCR17A, which encodes ibeA, was eluted from 1.0% agarose gel after digestion and then ligated to the same restriction sites of pET28a(+) (Novagen). Expression from this construct (pET17A) resulted in a 33-amino acid peptide (including the T7-Tag and 6×His-Tag) fusion to the N-terminus of IbeA. The size of the fusion protein was 53-kDa. BL21(DE3) carrying T7 RNA polymerase gene was used as the host strain for pET17A transformation. Transformants were identified by the predicted phenotypes (kanamycin resistance). The protein expression was induced with 0.5 mM IPTG at 37° C.

Purification of IbeA protein. Small-scale expression and purification of the recombinant protein was carried out according to the manufacturer's instructions (Novagen, Madison, Wis.). The protein preparations isolated from the supernatants and pellets were resolved on 10% polyacrylamide gel (SDS). Insoluble IbeA protein with a histidine tag was purified by binding to Ni-NTA resin in 8 M urea according to the manufacturer's instructions (Novagen, Madison, Wis.). The eluted proteins containing 8 M urea were refolded as described previously [Huang S H, et al, Infect Immun (1995); 63:4470-5.]. Purity of the final product was assessed by subjecting the indicated amount of protein to sodium dodecyl sulfate (SDS)-10% polyacrylamide gel electrophoresis (PAGE) by the procedure of Laemmli [Sambrook J, Fritsch E F, Maniatis T. Molecular cloning: a laboratory manual, 2nd ed. Cold Spring Harbor, New York; Cold Spring Harbor Laboratory Press, 1989:1.1-18.57]. Protein concentration was determined by using the Bio-Rad protein standard assay reagents according to the manufacturer's instruction. The protein was examined for the effect on the invasion of BMEC by the strain E44. Briefly, BMEC was incubated with the purified IbeA protein or bovine serum albumin (negative control) for 1 hr (0.125 to 1.5 μg/well) at room temperature before adding bacteria and invasion assays were carried out as described above.

### Example 1

Isolation and subcloning of large DNA fragment carrying the full-length ibeA gene. Escherichia coli K1 strain RS218 was used as the source of DNA for cloning experiments. This strain has been shown to invade BMEC and induce meningitis in newborn rats [Huang S H, et al, Infect Immun (1995); 63:4470-5]. To clone the invasion determinants from RS218, a genomic library was constructed in lambdaGEM-12. Using the partial sequence of ibeA (0.58 kb) as a probe, approximately 5×10⁵ recombinant phages were screened and two phage clones for ibeA were identified. The recombinant phage DNAs were purified and digested with NotI. The sizes of the inserts were between 16 and 18 kb. An 18-kb insert containing ibeA was subcloned into pUC13 (pUC1030) and pWKS30 (pWKS1030).

### Example 2

Sequence Analysis of ibeA. In order to further define the full-length ibeA gene, we sequenced the 2.3 kb SphI fragment carrying the full-length ibeA gene. A single ORF was identified in this region. As shown in FIG. 1, a 1,368-nucleotide open reading frame (ORF) assigned to the ibeA gene coded for a protein with 456 amino acids and a calculated molecular mass of 50 kDa (FIG. 1). A minor sequence error in the partial gene has been corrected. No significant sequence homology was observed between ibeA and other known genes when DNA and protein databases in GenBank were searched. Potential -10 (CTTATA) and -35 (GTTAAT) promoter regions were found at the 5′ noncoding region of ibeA.

### Example 3

Nucleotide sequence accession number. The complete nucleotide sequence of ibeA has been deposited in the GenBank Nucleotide Sequence Data Library under the GenBank accession number AF289032.

### Example 4

Determination of the size of IbeA protein in the in vitro transcription/translation and in vivo protein expression systems. The size of IbeA protein was verified by in vitro protein expression experiments performed in an Escherichia coli T7 S30 extract assay system. In this system in vitro transcription and translation of the DNA fragments containing ibeA gene were coupled. Initially, pWKS1030 carrying an 18 kb ibeA locus and several restriction fragments derived from pWKS1030 were tested in this system. A 50 kDa protein was only synthesized in pWKS1030 (data not shown) and pFN23A carrying a 2.3 kb SphI fragment, suggesting that the full-length ibeA gene most likely resides in this region. As shown in FIG. 2A, the reaction mixtures with the recombinant plasmid pFN23A containing the full length ibeA gene synthesized a 50 kDa protein, whereas the reaction mix with the vector alone did not synthesize such proteins. Synthesis of a 50 kDa protein by the ibeA construct in the in vitro translation system suggests that the size of IbeA protein is 50 kDa. For further confirmation of the size of IbeA, pFN23A was used for in vivo protein expression [Sankar P, et al, J Bacteriol (1993); 175:5145-52]. This expression system minimizes toxic gene effects under timing control of T7 RNA polymerase. The same size (50 kDa) of protein products was shown on the SDS-PAGE gel (FIG. 2B). This protein band was isolated and subjected to N-terminal sequencing. The N-terminal amino acid sequence of IbeA was indicated in FIG. 1. The size and N-terminal sequence of the expressed protein in vitro and in vivo perfectly matched that of the ORF deduced from DNA sequence data (FIG. 1).

### Example 5

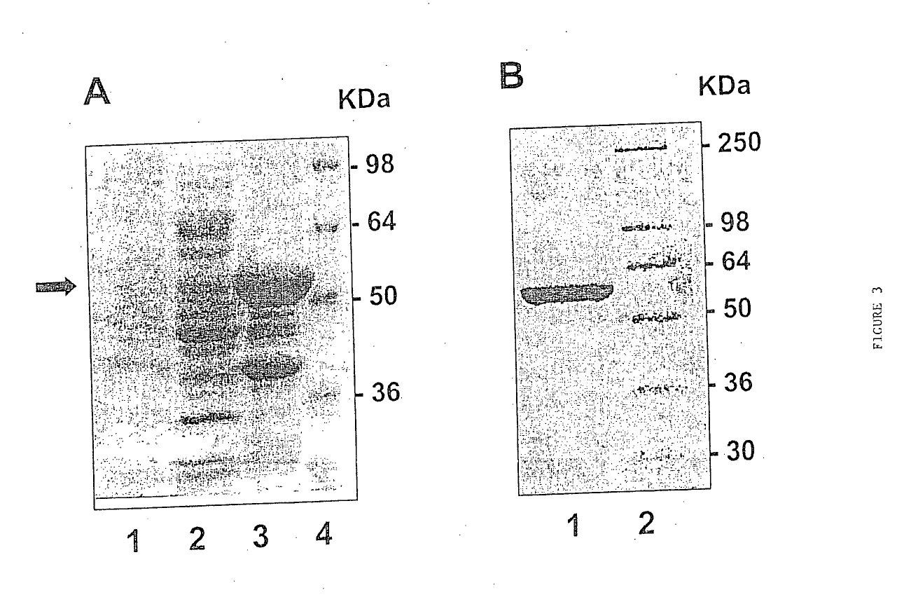

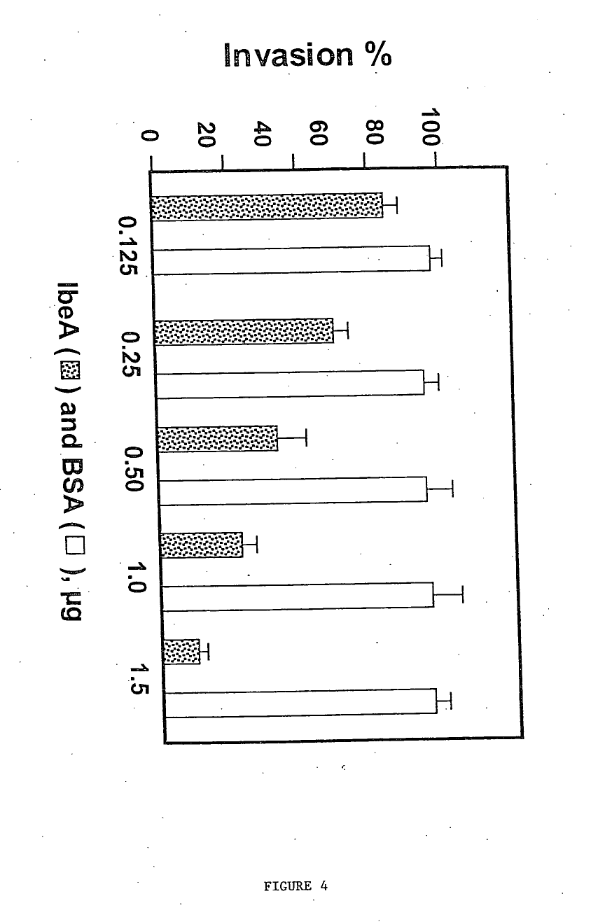

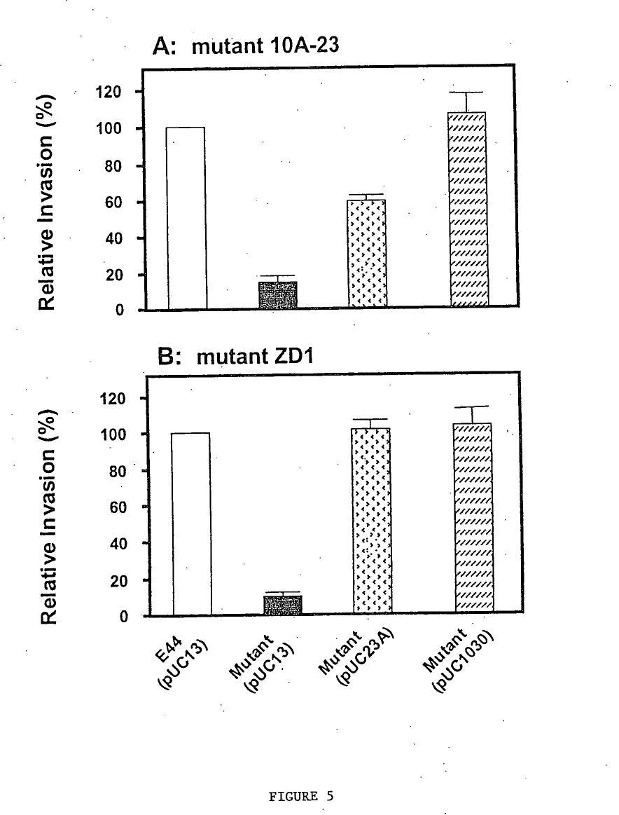

Blocking Escherichia coli K1 Invasion of BMEC with the purified IbeA: The ibeA ORF of the DNA fragments cloned in this work was expressed in Escherichia coli BL21 (DE3)/pET28a expression system to examine the function of the expression product. This construct expresses a fusion protein (His₆-IbeA) that is composed of a His₆ tag and the entire ibeA ORF (50 kDa). When this authentic His₆-IbeA fusion protein was expressed in Escherichia coli strain BL21 (DE3), a 53-kDa protein was present in the inclusion body (FIG. 3A). The His₆-IbeA protein was purified by Ni-NTA affinity chromatography from the insolube fraction (FIG. 3A). Refolding resulted in a soluble IbeA migrating with a single band of the same size (FIG. 3B). The function of this protein was tested by examining the purified recombinant IbeA protein for the ability to inhibit invasion of BMEC by strain E44. 0.125 to 1.5 μg of the purified IbeA was pre-incubated with BMEC for 1 h at room temperature. The bacteria were added to the confluent monolayer of BMEC for invasion assay. As shown in FIG. 4, the IbeA protein effectively inhibited Escherichia coli K1 invasion of BMEC in a dose-dependent manner, whereas the control protein BSA showed no such inhibition. Neither IbeA nor BSA affected bacterial viability under the experimental conditions used. More importantly, the entire recombinant IbeA protein showed much higher efficiency (50 times) in blocking the invasion of BMEC by Escherichia coli K1 than the partial protein fragment [Huang S H, et al, Infect Immun (1995); 63:4470-5], indicating that functional activities are greater with the whole protein.

### Example 6

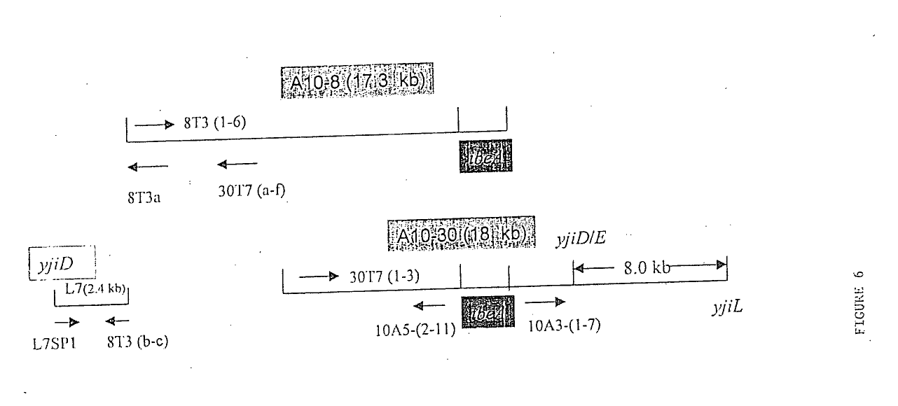

Construction and complementation of the ibeA-deletion mutant ZD1. To address the question whether the non-invasive phenotype of the TnphoA mutant 10A-23 is due to a polar effect, we constructed a mutant, ZD1 carrying 95% deletion of the ibeA ORF. The deletion of ibeA was confirmed by PCR and DNA sequencing. The non-invasive phenotypes of 10A-23 and ZD1 were shown in FIG. 5. Both pUC23A carrying the entire coding region of ibeA gene and pUC1O30 with a larger ibeA locus were able to completely restore the invasion ability of the non-invasive mutant ZD1 (FIG. 5B). pUC1030 was able to completely complement the TnphoA insertion mutant 10A-23, while pUC23A was capable of partially restoring the non-invasive phenotype of 10A-23 (FIG. 5A), suggesting that additional downstream gene(s) may be affected by the TnphoA insertion. However, pUC1030 is not sufficient to complement non-pathogenic Escherichia coli K12 strain HB101 (data not shown). These data provided further evidence to show that ibeA gene is one of the major determinants contributing to Escherichia coli K1 invasion of BMEC.

TABLE 1
Escherichia coli K1 (meningitis-associated) or K12 (non-pathogenic)
Strains, and Plasmids Used in this study.

| | Characteristics | Reference |
|---|---|---|
| Strains | | |
| RS218 | O18:K1:H7 | (1) |
| | (causing 32% of Escherichia coli meningitis) | |
| E44 | RS218, Rif^{R} | (1) |
| 10A-23 | E44 ibeA::TnphoA | (2) |
| ZD1 | ΔibeA derivative of E44 via allelic exchange | This study |
| DH5α (λpir) | K 12 strain | (3) |
| SM10 (λpir) | K 12 strain | (4) |
| Plasmid | | |
| pFN476 | Amp^{r}, low copy, T7 promoter | (5) |
| pFN23A | pFN476 carrying ibeA gene (2.3 kb) | This study |
| pUC13 | Ampr, lacZ | (6) |
| pWKS30 | Ampr, lacZ | (7) |
| pWKS1030 | pWKS30 carrying ibeA locus (18 kb) | This study |
| pUC1030 | pUC13 carrying ibeA locus (18 kb) | This study |
| pUC23A | pUC13 carrying ibeA gene (2.3 kb) | This study |
| pET28a(+) | Kan^{r}, F1 origin, His•Tag | Novagen |
| pCR17A | pCRII carrying ibeA gene (1.7 kb) | This study |
| pET17A | pET28a(+) carrying ibeA gene (1.7 kb) | This study |
| pCVD442 | Amp^{r}, oiRr6K, sacB, mobRP4 | (3) |
| pVZD | pCVD442 carrying DNA with ΔibeA | This study |

References for Table 1:
(1) Hoffman JA, et al, Infect Immun (2000); 68: 5062-7; Huang SH, et al, Infect Immun (1995); 63: 4470-5; Huang SH, et al, Infect Immun (1999); 67: 2103-9;
(2) Huang SH, et al, Infect Immun (1995); 63: 4470-5;
(3) Donnenberg MS, et al, Infect Immun. (1991); 59: 4310-7;
(4) Donnenberg MS, et al, Infect Immun (1990); 58: 1565-71; Donnenberg MS, et al, Infect Immun. (1991); 59: 4310-7;
(5) Sankar P, et al, J Bacteriol (1993); 175: 5145-52;
(6) Wang Y, et al, Infect Immun (1999); 67: 4751-6;
(7) Wang RF, et al, Gene (1991); 100: 195-9

TABLE 2

Oligonucleotides used for cloning, sequencing
and making the deletion mutant of ibeA gene.

| | Character- | |
|---|---|---|
| Strains | istics | Reference |
| 10A5-4a | + | 5′-TTGATCCCCGTACGCTTTC-3′ |
| 10ZM6 | − | 5′-ACGCGTGGGTTCCAGATAAAATTCC-3′ |
| 10D5M | + | 5′-AGACGCGTCAGGAACGCTTACAGC-3′ |
| 10D3 | − | 5′-CAAACCATCAGAACCGG-3′ |
| 10A5-3S | + | 5′-CTTGTACTCGGGTTAGAG-3′ |
| 10B3-4a | − | 5′-ATAACACCGATGCCAAC-3′ |
| 10A5-3Sa | + | 5′-AGTCGACTTGTACTCGGGTTAGAG-3′ |
| 10A3-1S | + | 5′-GTCGACATATGTTTAGCCCTTATC-3′ |
| 10A3-4a | − | 5′-GCAGTGTACCTGCATAG-3′ |
| 10A3-2 | + | 5′-TGAACGTTGTCAGCATC-3′ |
| 10A3-3 | + | 5′-CCCTAATGCCAACAATC-3′ |

### DISCUSSION

Current evidence suggests that microbial penetration across the BBB and entry into the CNS are a result of the specific interaction of several bacterial determinants with BMEC, which is a major component of the BBB [Huang, S H, et al, Microbes and Infection (2000); 2:1237-44. Kim K S, et al, Subcell Biochem (2000); 33:47-59]. Our investigations of this issue with Escherichia coli have shown that successful traversal of Escherichia coli across the blood-brain barrier requires two independent steps of bacterium-BMEC interactions, i.e., Escherichia coli binding to BMEC and invasion of BMEC. We have previously shown that S fimbriae contribute to Escherichia coli binding to BMC [Stins W F, et al, Am J Pathol. (1994); 145:1228-36]; however, binding to BMEC via S fimbriae was not accompanied by invasion of BMEC. We have subsequently identified several Escherichia coli K1 determinants contributing to invasion of BMEC, i.e., IbeA, IbeB, YijP, AslA, and OmpA [Hoffman J A, et al, Infect Immun (2000; 68:5062-7. Huang S H, et al, Infect Immun (1995); 63:4470-5. Huang S H, et al, Infect Immun (1999); 67:2103-9. Wang Y, et al, Infect Immun (1999); 67:4751-6. Prasadarao N V, et al, Infect Immun (1996); 64:146-51]. IbeB, yijP, aslA, and ompA have homologues present in the Escherichia coli K12 genome, while the ibeA gene was found to be a specific virulence factor present in CSF isolates of Escherichia coli K1 [Huang S H, et al, Infect Immun (1995); 63:4470-5. Bingen E, et al, J Infect Dis (1998); 177:642-50. Bonacorsi S P, et al, Infect Immun (2000); 68:2096-101].

We have previously shown that the TnphoA mutant of ibeA(10A-23) was significantly less invasive in BMEC monolayers in vitro as well as in the newborn rat model of hematogenous meningitis than the parent strain. A small recombinant protein fragment (8.2 kDa) encoded by the partial ibeA gene was able to block Escherichia coli K1 invasion of human BMEC [Huang S H, et al, Infect Immun (1995); 63:4470-5]. In further characterization of the ibeA gene, we have several lines of evidence showing that the product encoded by the full-length ibeA gene was a 50-kDa protein. The first evidence that IbeA is synthesized as a 50 kDa protein came from transcription/translation experiments performed in an Escherichia coli T7 S30 extract assay system. This data was further supported by the detection of the same size of a gene product by expression and purification of the IbeA protein from in vivo protein expression systems. The first 20 residues of the N-terminal sequence of the purified protein completely matched that of the deduced ORF from the DNA sequence data. These results indicate that the size of the IbeA protein is 50 kDa. The deduced protein sequence suggests that IbeA is a potential membrane protein with three transmembrane domains (FIG. 1). However, no significant homology was found with known genes including any other recognized invasion proteins, suggesting that IbeA is unique to the known microbial virulence proteins.

As described previously, ibeA gene was initially identified by TnphoA mutagenesis [Huang S H, et al, Infect Immun (1995); 63:4470-5]. In order to exclude the possibility that a non-invasive property of 10A-23 is related to a polar effect of TnphoA on the other genes involved in invasion, an isogenic deletion mutant of ibeA, ZD1, was made and tested for its invasion phenotype in BMEC. Like the TnphoA mutant 10A-23, the deletion mutant ZD1 was less invasive in BMEC. The ibeA gene encoding the 50 kDa IbeA protein was able to partially and completely restore the non-invasive phenotype of 10A-23 and ZD-1 mutants, respectively. However, pUC1O30 carrying a 18 kb ibeA fragment was capable of completely restoring the invasion ability of both 10A-23 and ZD1 to the level of the parent strain E44, suggesting that additional downstream gene (s) might be affected by the TnphoA insertion in the ibeA gene. Furthermore, Escherichia coli K12 strain HB101 was not complemented by pUC1030 (data not shown), implying that additional determinants may contribute to internalization and intracellular survival of Escherichia coli in BMEC. It remains to be determined how multiple different invasive determinants of Escherichia coli K1 contribute to crossing of the blood-brain barrier.

We have recently identified a 55 KDa membrane protein from BMEC, which binds to a partial fragment of IbeA (8.2 kDa). A soluble form of this IbeA receptor (IbeAR) and a polyclonal antibody against the IbeAR block Escherichia coli K1 invasion of BMEC [Prasadarao N V, et al, Infect Immun (1999); 67:1131-8]. We have previously shown that the partial IbeA fragment was able to inhibit Escherichia coli K1 invasion of BMEC but high dose (50 μg) was required for approximately 80% inhibition [Huang S H, et al, Infect Immun (1995); 63:4470-5]. However, as shown in this report, we demonstrated that the entire recombinant 50 kDa IbeA protein was much more efficient in blocking Escherichia coli K1 invasion of BMEC and only 1 μg dose achieved a similar degree of inhibition. Taken together, these findings suggest that the previously reported 8.2 kDa fragment of IbeA represents a part of the binding domain interacting with IbeAR, but other structures of IbeA are required for optimal interaction with BMEC.

In conclusion, our data further support that the ibeA is a major determinant for Escherichia coli K1 invasion of BMC, an essential step in the pathogenesis of Escherichia coli meningitis. Studies are in progress to define the mechanisms by which ibeA locus contributes to Escherichia coli K1 invasion of the blood-brain barrier.

### Example 7-12

In order to identify E. coli structures that contribute to the invasion of the blood-brain barrier (BBB), we have used transposon TnphoA mutagenesis to generate a collection of noninvasive mutants. Four noninvasive mutants, 10A-23, 7A-33, 23A-20, and 27A-6 with a single TnphoA insertion and without any changes in other phenotypic and genotypic characteristics, were found to be significantly less invasive of BMEC monolayer in vitro and of the central nervous system in the newborn rat model of hematogenous E. coli meningitis (Huang et al., (2000). Microbes and Infection. 2:1137-44; Huang, and Jong (2001) Cellular Microbiology; Huang et al., (2001) J. Infect. Dis. 183:1071-8; Hoffman, J. A. et al (2000) Infect. Immun. 68:5062-5067). Accordingly, four E. coli genetic determinants ibeA, ibeB, yijP, and aslA contributing to E. coli invasion of the BBB have been identified. IbeB, yijP and aslA have homologues present in the non-pathogenic E. coli K-12 genome. However, the ibeA gene encoding a 50-kDa protein has been found to be unique in cerebrospinal fluid (CSF) isolates of E. coli K1 (e.g., C5 and RS218), while laboratory strains of E. coli K-12 (e.g., DH5 and HB101), as well as noninvasive E. coli (e.g., E412), lack ibeA (Huang. et al., (1995) Infect. Immun. 63:4470-4475; Huang et al., (2001) J. Infect. Dis. 183:1071-8; Johnson, J R et al., (2001) J Infect Dis. 183:425-34). The ibeA locus was able to completely complement the TnphoA insertion and isogenic deletion mutants 10A-23 and ZD1 (Huang et al., (2001) J. Infect. Dis. 183:1071-8.

We report here a 20.3-kb locus including the ibeA gene that is found to be unique in E. coli K-1 strains. It is situated between yjiD and yjiE, adjacent to the fim operon, and absent in non-pathogenic E. coli strains. It has regions different in G+C content from the rest of the genome, indicating that this gene cluster is a genetic island of meningitic E. coli containing ibeA (gimA). Additional 14 novel open reading frames (ORFs) have been identified.

### Materials and Methods for Examples 7-12

Bacterial strains, plasmids and media. E. coli strain RS218 (018:K1:H7) is a clinical isolate from the CSF of a neonate with meningitis (Huang, S. H. et al., (1995) Infect. Immun. 63:4470-4475). E44 is a spontaneous rifampin-resistant mutant of RS218, which has been characterized (Huang, S. H. et al., (1995) Infect. Immun. 63:4470-4475; Huang, S. H. et al., (1999) Infect. Immun. 67:2103-2109; Huang, S H et al., (2001) J. Infect. Dis. 183:1071-8.). DH5α was used as the host strain in subcloning and preparation of plasmids for DNA sequence determination. Strains containing plasmids were grown at 37° C. in L broth with ampicillin (100 μg/ml), kanamycin (50 μg/ml) for positive selection of plasmids. Bacteria were cultured in L broth and stored in L broth with 20% glycerol at −70° C.

Chemicals and enzymes: Restriction endonucleases, T4 DNA ligase, and other enzymes were purchased from New England Biolabs (Beverly, Mass.,) unless otherwise noted. Chemicals were purchased from Sigma (St. Louis, Mo.). All isotopes were obtained from New England Nuclear Corp. (Boston, Mass.,). Reagents for preparation of DNA sequencing gels were ultra pure quality from National Diagnostics (Atlanta, Georgia). The reagents for DNA sequencing reaction with Sequenase and other chemicals were purchased from United States Biochemical Corp. (Cleveland, Ohio). DNA sequencing kits with dye terminators were obtained from PE Applied Biosystem (Great Britain).

Extraction and manipulation of plasmids and subcloning. All genetic manipulations were performed by standard methods (Sambrook, J., et al., (1989). Molecular Cloning: a Laboratory Manual, 2nd ed. Cold Spring ). Plasmid All isotopes were obtained from New England Nuclear Corp. (Boston, Mass.). DNA was extracted by using a Plasmid Mini kit (Qiagen Inc., Chatsworth, Calif.). Purification of DNA fragments and extraction from agarose gel slices were performed with GeneClean™ (Bio 101, La Jolla, Calif.). Competent cells of E. coli were made in 10% glycerol and transformed with electroporation (Huang, S. H. et al., (1999) Infect. Immun. 67:2103-2109; Huang S H et al., (2001) J. Infect. Dis. 183:1071-8). Genomic DNA was partially digested with SauA3 I (New England Biolabs), which is compatible with BamHI. Partially digested genomic DNA (15 to 23 kb) was partially filled-in with dGTP and dATP. This DNA was ligated into LambdaGEM™-12 arms with XhoI Half-site. Ligation and packaging of recombinant lambda phage were performed according to the manufacturers instructions (Promega). The E. coli genomic library was screened by DNA hybridization (Huang et al, 1999, 2001) to identify phage clones that contained ibeA. A 0.58-kb ibeA DNA fragment in pCIB10B (Huang et al 1995) was released with EcoRI and purified by preparative agarose electrophoresis and Geneclean (Bio101), labeled with [α³²P]dCTP by using an oligolabeling kit (Pharmacia), and used as a probe in the screening (>1×10⁸ cpm/μg). The phage plaques were replicated onto Nylon filters, UV-linked and hybridized as described previously (Huang et al 1999, 2001). Plaques hybridizing to the probe were identified by autoradiography and then purified.

Screening of E. coli RS218 genomic library by DNA hybridization. A genomic library of E. coli RS218 was constructed as previously described (Huang, S H et al., (2001) J. Infect. Dis. 183:1071-8). The E. coli genomic library was screened by DNA hybridization to isolate phage clones that contained the ibeA locus. A 0.58 kb ibeA DNA fragment was purified by preparative agarose electrophoresis and GeneClean™ (Bio101), labeled with [³²P]dCTP by using an oligolabeling kit (Pharmocia), and used as a probe in the screening (>1×10⁸ cpm/μg). The phage plaques were replicated to Nylon filters, UV-linked and hybridized as described previously. Plaques hybridizing to the probe were identified by autoradiography, and then purified.

PCR. PCR was carried out by a standard method. The left junction region was amplified by YjD1 (5′-AAT GCT GTA CCA CGA CG-3′) and 8T3a (5′-T CAT AGT CTA CGT CTC GCC GAC-3′), using 30 cycles of 94° C. for 1 min, 58° C. for 1 min, 70° C. for 3 min.

Preparation of templates for nucleotide sequencing. Two overlapping genomic clones (10-8 and 10-30) and a PCR clone, prepared from genomic DNA from E. coli RS218 and found previously to carry a DNA sequence specific to E. coli K1 strain, were used for nucleotide sequencing through primer walking.

DNA sequencing and analysis: At first the partial DNA sequence of the gimA was determined by the dideoxy chain termination method of Sanger et al with Sequenase version 2.0 kit from U.S. Biochemicals Corp. (Cleveland, Ohio) and Taq DNA polymerase. DNA sequence analysis was performed manually with [³⁵S]dATP(1,000 to 1,500 Ci/mmol) obtained from Du Pont NEN Research Products (Boston, Mass.). Subsequently we switched to the automatic DNA sequencing approach with fluorescence labeled nucleotides (373A ABI automated sequencer) because of higher speed, lower cost and avoiding electrophoretic compression artifacts derived from the high G +C content. The entire ibeA gene cluster was sequenced by primer-walking. Both strands of the DNA were sequenced to ensure the accuracy.

Bioinformatics Approaches

The sequence data was analyzed with the DNA analysis programs developed by Scientific & Education Software (Durham, N.C.). DNA and deduced protein sequences were used to search the DNA and protein data bases at the National Center for Biotechnology Information (National Library of medicine, Washington, D.C.) by using the BLAST algorithm. ClustalW (Thompson J D et al (1994) Nucleic Acids Res. 22:4673-4680) and Boxshade (Hofmann K et al., (1999) Nucleic Acids Res. 27:215-9) were used for multiple alignment of protein sequences. Annotation of genes from the genetic island gimA makes use two conceptually different approaches. First, sequences are compared to existing sequences with known functions at the protein level to identify homologous regions and then to predict the functionality of the novel genes. Second, a uniform Hidden Markov Model for annotation of prokaryotic genes is used to predict the unknown genes in gimA (Shmatkov A M et al., (1999) Bioinformatics 15:874-86). Since tightly packed prokaryotic genes frequently overlap with each other, it is easy by targeting gene starts and overlapping genes to makes detection of translation initiation sites and, therefore, exact predictions of prokaryotic genes. A combination of the comparative analyses and the ‘frame-by-frame’ algorithm leads to annotation of a given novel gene. Phylogenetic analysis was performed by using BioNavigator (http://www.bionavigator.com).

### Example 7

Cloning and subcloning of the ibeA gene cluster of E. coli RS218.

E. coli K1 strain RS218 was used as the source of DNA for cloning experiments. This CSF isolate was capable of invading human and bovine brain microvascular endothelial cells (BMEC) and inducing meningitis subsequent to bacteremia in newborn rats. To clone the ibeA determinants from RS218, a genomic library was constructed in LambdaGEM-12. By using half sites of XhoI in the vector and SauA3I in the genomic inserts for library construction, self-ligation of vector and genomic sequences was eliminated since only recombinant phages containing a single insert of the appropriate size (15-23 Kb) were capable of being packaged. Approximately 5×10⁵ recombinant phages were screened by using an ibeA fragment (0.58 Kb) as a probe. Three overlapping phage clones for ibeA were identified. The recombinant phage DNAs were purified and digested with NotI. The size of the two overlapping inserts was 17.3 (A10-8) and 18.0 (A10-30) Kb, respectively. These inserts containing ibeA gene cluster were subcloned into NotI site of pBluescript KS (pKS108 & pKS1030).

### Example 8

Identification of the Junction Regions.

The right junction (where “right” is defined as the sequences associated with higher numbers on the linkage map of E. coli K12) was identified by T3 primer on the A10-30 clone. A stretch of 530 bp sequence was determined. A search of GenBank revealed that this region is identical to the C-terminal coding sequences of yjiD (12 bp) and yjiE (518 bp) in E. coli K12. The gimA insertion site is located inside yjiD ORF, 9-bp upstream of yjiD stop codon, and outside yjiE ORF, 4 bp upstream from yjiE stop codon. 18-kb sequences were obtained from A10-8 and A10-30 clones. According to the physical mapping (Bloch et al. 1996), gimA is about 20 kb in size. In order to identify this sequence gap, a 5′-primer (10YjD1) and a 3′-pimer (8T3a) were picked up from 62 bp upstream of yjiD stop codon and the 5′ region of the known gimA sequence, respectively. To isolate and verify the left junction of gimA, these two primers were used for PCR to amplify this region directly from E44 genomic DNA. A 2.4 kb DNA was isolated and subcloned into pGEM-T easy vector (E44L7). Sequence analysis of this PCR clone defined the left junction and revealed that a 2.3 kb sequence is unique to E. coli K1 and has a 30 bp overlapping region with the known gimA sequence. Therefore, gimA is 20.3 kb in size.

### Example 9

Nucleotide Sequences of gimA.

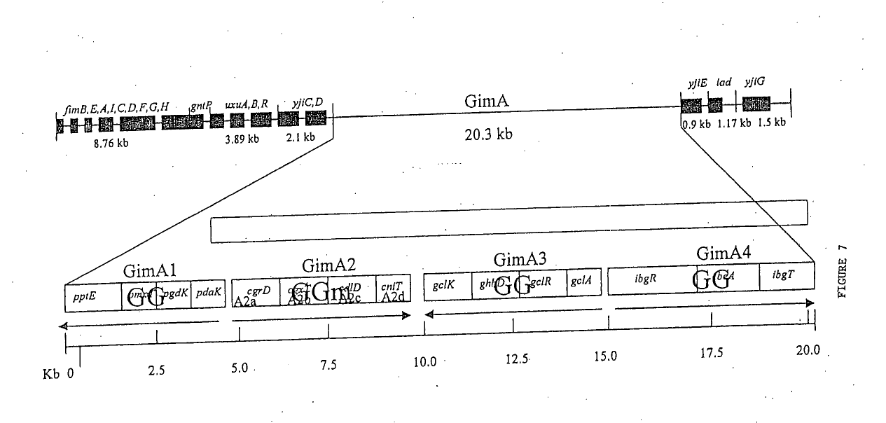

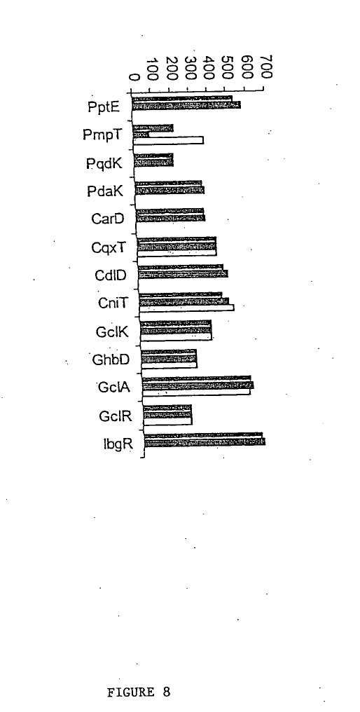

The DNA sequences of the ibeA gene cluster were determined by a primer walking approach with primers complementary to both strands of gimA genes in A10-8, A10-30 and E44L7. Analysis of the DNA sequence data indicated that gimA is located in 98 min. Two phage clones (A10-8 and A10-30) shared an overlap region in which they have the identical ibeA and the downstream region (FIG. 6). The results of open reading frame (ORF) analysis and the features of the gimA were summarized in FIGS. 7 and 8 and Table 4.

### Example 10

Novel Genes Inside the gimA.

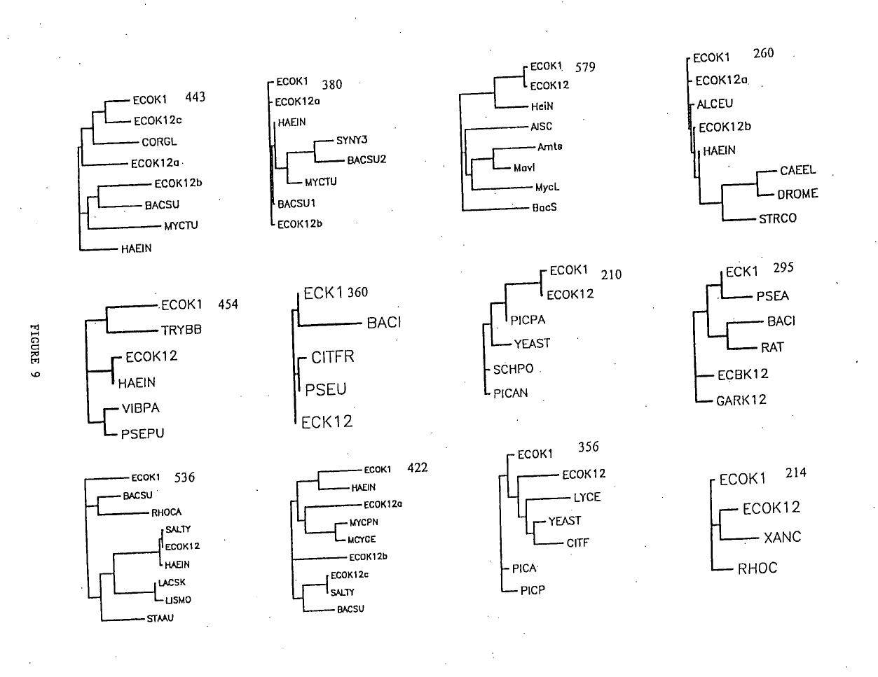

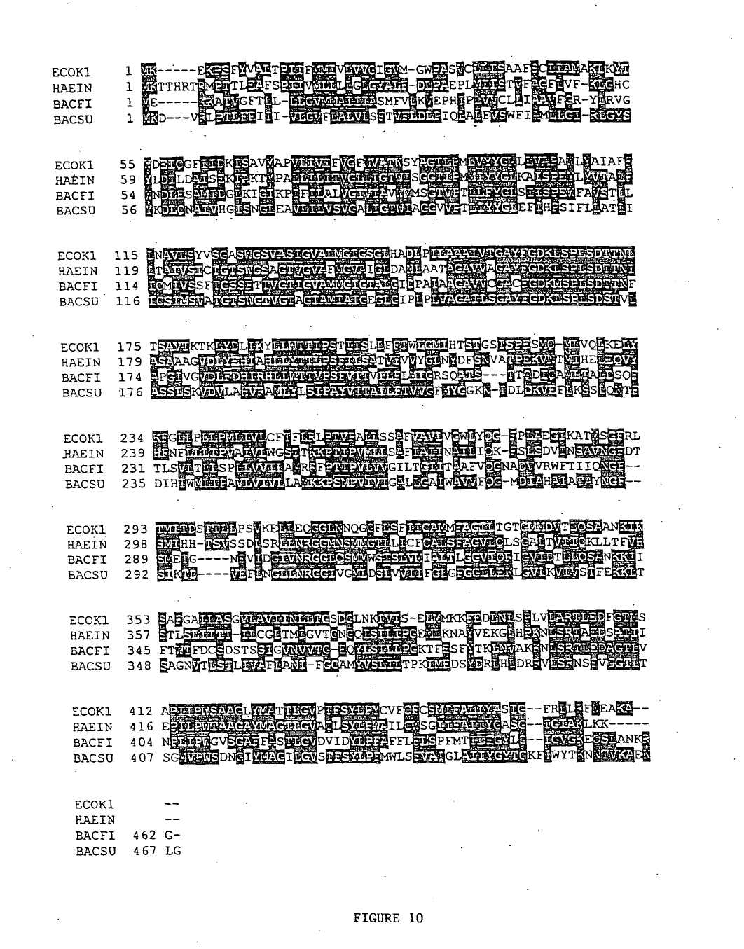

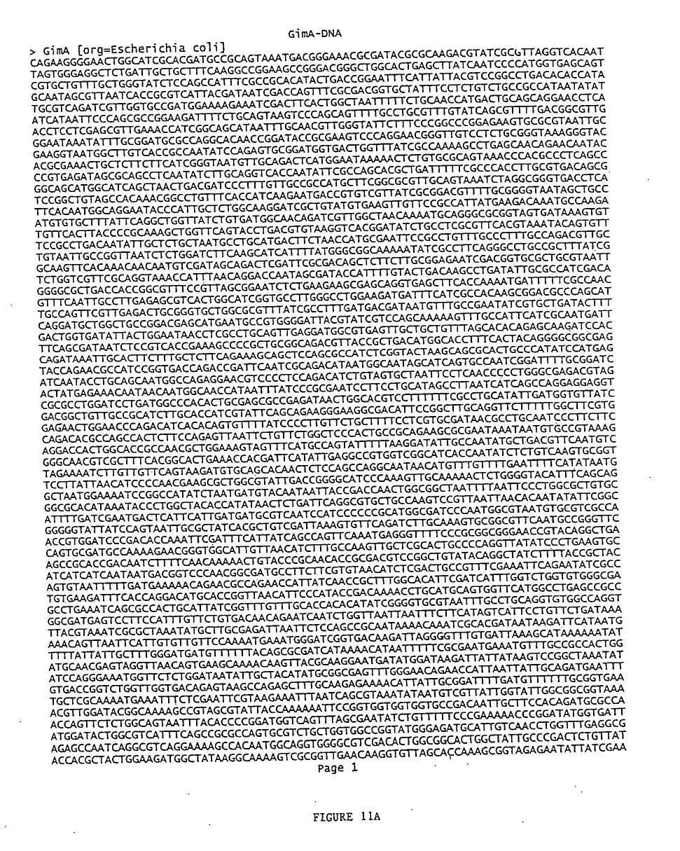

To determine ORFs present within the gimA, the entire gene cluster was subjected to nucleotide sequencing. In this region, which included an apparent 20.3-kb gimA, we have identified 15 ORFs including the known gene ibeA (FIG. 9). Among the sequenced ORFs are genes that appear to be involved in carbohydrate metabolism, transport systems, protein binding and transcriptional regulation (Table 4).

### Example 11

Insertion sequences and transposons. No known insertion sequences have been identified from gimA and its junctions. However, a search of GenBank inidicated that a 57 bp region (5358-5414) shares significant homology (85%) with transposon Tn5542 in Pseudomonas putida.

### Example 12

Other features of the gimA. The gimA of strain RS218 displays two other features common to such blocks of virulence genes. First, the GC content of the sequences, not including those of K-12 origin, is 46.2%. This value is significantly different from a value of 50.8% for the E. coli genome. Also, the gimA sequence is unique to E. coli K1 strain and does not share significant homology with sequences of K-12 origin.

### DISCUSSION

We have characterized a 20.3-kb region of DNA from E. coli RS218, a CSF isolate from a newborn baby with E. coli meningitis, by isolation of overlapping phage and PCR clones, restriction endonuclease mapping, subcloning, and DNA sequencing (FIG. 7). In this region, we have identified what can be defined as a genetic island that includes 15 ORFs (Table 4 and FIG. 9). Genetic or pathogenicity islands typically have a GC content lower than that of neighboring DNA, and have gene clusters positioned near each other which contribute to a single virulence property. The gimA of RS218 is 20.3 kb in size (FIG. 8), has a GC content of 46.2% (compared to 50.8% in K-12 genomic DNA).

Nucleotide sequences of the 20.3-kb gimA of RS218 reveal 14 newly described genes. The ibeA gene has been found essentially in E. coli meningitis and significantly more frequently detected in strains that are positive for the sfa/foc operon than in strains that are negative for the sfa/foc locus. Therefore, we postulate that these newly described genes along with ibeA may represent a genetic island that contributes to the pathogenesis of neonatal meningitis caused by E. coli. Insertion and deletion in ibeA gene led to a non-invasive phenotype of RS218 in vitro and in vivo, suggesting that this virulence determinant contributes to E. coli invasion of the blood-brain barrier (Huang, S. H. et al., (1995) Infect. Immun. 63:4470-4475; Huang, S H et al., (2001) J. Infect. Dis. 183:1071-8). As we continue our studies on the genetic island of RS218, we will select more mutants with phenotypes that may relate to virulence such as metabolite uptake and transcriptional regulation. We will undertake allelic exchange mutagenesis of specific genes and test these isogenic mutants by in vitro BMEC invasion assays and, in vivo, by using the neonatal rat model of meningitis. By creating such mutants, we hope to identify additional genes of gimA contributing to the virulence phenotype of meningitic E. coli. It is likely, however, that meningitic E. coli strain RS218 contains additional genetic islands. For two uropathogenic strains that have been studied closely, 536 and J96, each strain was found to contain two separate pathogenicity islands. For strain 536, pathogenicity islands of 190 and 70 kb are inserted at 97 min (within leuX) and 82 min (within selC), respectively. For strain J96, PAIs of 110 and >170 kb are inserted at 94 min (within pheR) and 64 min (within pheV), respectively. Mapping of non-invasive TnphoA mutants revealed that at least two putative genetic islands, including gimA and a cluster of 12 TnphoA insertions located at a 120 kb RS218-specific chromosomal segment, are present in RS218 genome (Bloch, C. A. et al., (1996) FEMS Microbiol. Lett.,144: 171-176).Bloch et al. 1996). Very recently, Bloch's lab (Rode C K, et al., (1999) Infect Immun. 67:230-236) and Bingen's group (Bonacorsi SP et al., (2000) Infect Immun. 68:2096-2101) detected several new additions in RS218 genome by using comparative macrorestriction mapping and representational difference analysis, respectively. That RS218 may have additional genetic islands of unknown size raises the possibility that a significant number of other virulence genes, not detected on the gimA described here, may also contribute to the virulence of this strain.

TABLE 3

Oligonucleotides used for cloning and
sequencing.

| Primer | Strand | Sequence |
|---|---|---|
| 8T3a | − | 5′-T CAT AGT CTA CGT CTC GCC CAG-3′ |
| 8T3b | − | 5′-TCA ACG AAC TGG CAA TGC TG-3′ |
| 8T3c | − | 5′-CTA TTA CCC CGC AAA ACG TC-3′ |
| 8T31 | + | 5′-GCA ACC ATA ATT TAT CCC GCG-3′ |
| 8T32 | + | 5′-CGG CCA TAT CTA ATG ATG TAC-3′ |
| 8T33 | + | 5′-GCT ATC TTT TAC CGC TAC ATC-3′ |
| 8T34 | + | 5′-GGA TGA TGT TTT TTA CAG CGC G-3′ |
| 8T35 | + | 5′-ACA CTG GCG GCA CTG GCT ATT G-3′ |
| 8T36 | + | 5′-AGC GAC TAA TGC TGA ACT TGG-3′ |
| 8T32a | − | 5′-TAT TTA TGT GCG CCG CAC AG-3′ |
| 8T33a | − | 5′-GAT GTA GCG GTA AAA GAT AGC-3′ |
| 8T3ba | + | 5′-AGG ATG GCG TGA GTT GCT GC-3′ |
| 8T3bb | + | 5′-CCA GCA TTG CCA GTT CGT TG-3′ |
| 10A5-2 | − | 5′-GGTATATTACGAGCGGG-3′ |
| 10A5-3 | − | 5′-ATCTTCAGCTGCTTTAGTTAG-3′ |
| 10A5-4 | − | 5′-CTTCACGACGTTTGCGC-3′ |
| 10A5-5 | − | 5′-AATTTTCCCACACCTTCT-3′ |
| 10A5-6 | − | 5′-CGGCGGAAATACGAATC-3′ |
| 10A5-7 | − | 5′-CAT GAC CTC AGC ATC AC-3′ |
| 10A5-8 | − | 5′-GGC GTG TGT ATT GGC ACA TC-3′ |
| 10A5-9 | − | 5′-TCT GAT GCT TGA AAA GCG CC-3′ |
| 10A5-10 | − | 5′-TGA CGA ATT TCA CGT ACC TG-3′ |
| 10A5-11 | − | 5′-TAA CAA CAC CAG ACA AGC CC-3′ |
| 10A5-4a | + | 5′-TTGATCCCCGTACGCTTTC-3′ |
| 10A5-5a | + | 5′-GGGCAATTAAATCCATCTCTCC-3′ |
| 10A5-8a | + | 5′-CAT GAC GGG CCA GAA TAT G-3′ |
| 10A5-9a | + | 5′-ACA GGC ATT AAT CCA GTG GC-3′ |
| 10A5-11a | + | 5′-ACA CTC CTG CGC GAC TTC-3′ |
| 10A3-4 | + | 5′-AATTTCAGCGGCGTTTTCC-3′ |
| 10A3-5 | + | 5′-AGTGATACCACCAACC-3′ |
| 10A3-7 | + | 5′-TGGCTGTATCAAGGTTTC-3′ |
| 30T71 | + | 5′-GAC TAT CTA ATT TCC CTT CAC CG-3′ |
| 30T72 | + | 5′-CCT TGA ACT TGT GCC AGT TC-3′ |
| 30T73 | + | 5′-TAT TCA ACA GGC GGG CAT TC-3′ |
| 30T73a | − | 5′-AAA GGA ACA TTC GAACCC GG-3′ |
| 30T7a | − | 5′-AAT TTA CCG ACC GCG CTG AGTC-3′ |
| 30T7b | − | 5′-CGG TAC TTA AAC TCA TCG CTA C-3′ |
| 30T7c | − | 5′-CAT AAC GTG AGA AGG CCA GC-3′ |
| 30T7d | − | 5′-GAC GCA CGG TGC AAT TTT GC-3′ |
| 30T7e | − | 5′-CAG TTT TTG CCC CAA TCC GC-3′ |
| 30T7f | − | 5′-CGC AAT CCG CAT TGT TTT GAG-3′ |
| 30T7Ba | + | 5′-GAA TCG TCG CCA TCA CAC TC-3′ |
| 30T7BB | − | 5′-AGC CGA AAT TAG CCA GTA CC-3′ |
| 30T7CB | + | 5′-AAA AAA GGT GTG GCA TGG GC-3′ |
| 30T7Da | + | 5′-TAT TTC CGC AGG CGT AGT TGC-3′ |
| L7SP1 | + | 5′-CCA TCG GCA GCA TAA TTT GC-3′ |

TABLE 4
Gene annotation of the ibeA gene cluster (gimA)

| | | Size | Start | Start | Accession | | |
|---|---|---|---|---|---|---|---|
| Operon | ORF | (aa) | Position | codon | Number | Homologous to | Functions |
| GimA1 | PptE | 536 | 1694C | GTG | AF289032 | BACST PEP-PPT | phosphoenolpyruvate-protein |
| (PTS) | | | | | | (213/542 = 39%); E. ccli PT1 | phosphoryltransferase (PEP- |
| | | | | | | (203/540 = 37%) | PPT)(enzyme I) |
| | PmpT | 214 | 2466C | ATG | AF289032 | E. coli hypothetical protein | multiphosphoryl transfer |
| | | | | | | YCGC (98/212 = 46%); | protein (MTP) |
| | | | | | | XANCP PTF1/MTP | |
| | | | | | | (46/182 = 25%) | |
| | PgdK | 210 | 3102C | ATG | AF289032 | CITFR gylcerone kinase | Putative gylcerone kinase |
| | | | | | | (53/199 = 26%) | |
| | PdaK | 356 | 4185C | ATG | AF289032 | E. coli hypothetical protein | Putative DHA kinase |
| | | | | | | YCGT (211/355 = 59%); | |
| | | | | | | PICAN DHA kinase | |
| | | | | | | (136/364 = 37%) | |
| GimA2 | CgrD | 360 | 4504 | ATG | AF289032 | Glycerol dehydrogenase | Glycerol dehydrogenase |
| (Glycerol | | | | | | CITFR (225/359 = 62%; E. | |
| metabo- | | | | | | coli (204/359 = 56%) | |
| lism) | CgxT | 422 | 5653 | ATG | AF289032 | E. coli hypothetical protein | Putative transporter |
| | | | | | | YIHN (137/414 = 33%); | |
| | CdlD | 454 | 6939 | ATG | AF289032 | E. coli dihydrolipoamide | Putative dihydrolipoamide |
| | | | | | | dehydrogenase | dehydrogenase? |
| | | | | | | (25/44 = 56%) | |
| | CniT | 443 | 8604 | ATG | AF289032 | E. coli GAIT | Putative carnitine transporter |
| | | | | | | (130/404 = 32%) BACSU | (CAIT) |
| | | | | | | glycine betaine transporter | |
| | | | | | | (123/366 = 34%) | |
| GimA3 | GcxK | 380 | 11344C | ATG | AF289032 | E. coli hypothetical protein | Glycerate kinase (glxK) |
| (enolas | | | | | | YBBZ (171/372 = 45%) | |
| esuper- | GcxR | 295 | 12306C | ATG | AF289032 | E. coli hypothetical protein | Tartronate semialdehyde |
| family) | | | | | | YBBQ (166/290 = 57%); | reductase (glxR) |
| | | | | | | PSEAE HIBADH | |
| | | | | | | (85/249 = 34%) | |
| | GclA | 579 | 14858C | ATG | AF289032 | E. coli glycoxylate | Glycoxylate carboligase (gcl) |
| | | | | | | carboligase (393/578 = 67%) | |
| | GhyI | 260 | 13106C | ATG | AF289032 | E. coli glycoxylate induced | Hydroxypyruvate isomerase |
| | | | | | | protein & hydroxypyruvate | (hyi/gip) |
| | | | | | | isomerase (137/254 = 53%) | |
| GimA4 | IbgR | 624 | 15237 | ATG | AF289032 | DHAR Glycerol metabol. | Putative GMO regulatory |
| (regulation | | | | | | Operon (GMO) regulator | protein |
| of | | | | | | (186/590 = 31%) | |
| gimA) | IbeA | 456 | 17476 | ATG | AF289032 | DROME AMAN II | Invasion protein contributing |
| | | | | | | (27/97 = 27%); PLAVS | to E. coli invasion of the |
| | | | | | | erythrocyte binding protein | blood-brain barrier in vitro |
| | | | | | | (18/64 = 28%) | and in vivo; binding to IbeA |
| | | | | | | | receptor on brain endothelial |
| | | | | | | | cells. |
| | IbgT | 467 | 18861 | ATG | AF289032 | HAEIN Na(+)/H(+) | Putative Na(+)/H(+) |
| | | | | | | antiporter (148/435 = 34%) | transporter |

C = complementary strand

### Example 13

Induction of Apoptosis by IbeA

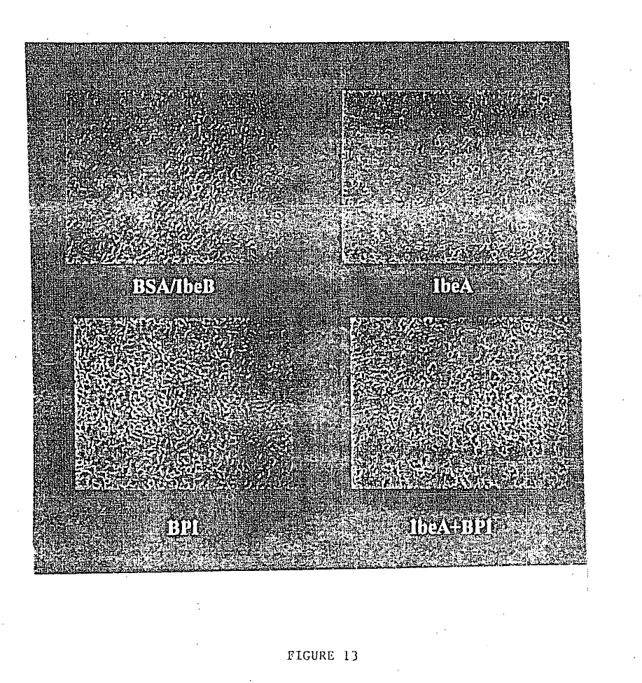

Several studies suggested that hippocampal apoptosis was a characteristic feature of bacterial meningitis in human disease and in several animal models of meningitis (Nau R et al., J Neuropathol Exp Neurol. 1999, 58(3):265-74; Loeffler J M et al., J Infect Dis. 2001, 183(2):247-252.; Bottcher T et al., J Infect Dis. 2000, 181(6):2095-8; Braun J S et al., Nat Med. 1999, 5(3):298-302; Leib S L et la., J Clin Invest. 1996, 98(11):2632-9). As shown in our previous studies, IbeA-dependent E. coli K1 invasion was observed in both intestinal epithelial and brain endothelial cells. Since apoptosis is a key feature of bacterial meningitis, we proposed that apoptosis might be associated with E44 invasion. In order to determine whether apoptosis plays a role in pathogenesis of E. coli meningitis, we tested apoptotic activity of IbeA and IbeB proteins. Human EC were grown on collagen coated eight-well chamber slide. At confluence, human EC were treated with 5 μg of protein [purified E. coli protein IbeA, IbeB, BPI or bovine serum albumin (IbeB and BSA are used as control)] and incubated for up to 6 hrs at 37° C. Subsequently, human EC were washed three times with experimental medium, fixed with 1% paraformaldehyde in PBS at 4° C. (Stins, M. F. et al., J. Neurovirol. In press). Apoptotic cells were detected by ApopTag in situ apoptosis detection kit (Intergen, Purchase, N.Y.), according to the manufacturer's instructions. Briefly, the 3′-OH DNA ends that were generated by DNA fragmentation become substrates for terminal deoxynucleotidyl transferase (TdT) and then digoxigenin nucleotides were catalytically added to the apoptotically produced DNA ends. These nucleotides were detected by antidigoxigenin antibody carrying a conjugated peroxidase. Diaminobenzidine was then reacted with peroxidase to produce insoluble brownish-colored products in apoptotic bodies where DNA fragmentations were present. The slides were then viewed and photographed in the microscope (Stins, M. F. et al., J. Neurovirol. In press.). As shown in FIG. 13, IbeA was capable of inducing apoptosis in human EC comparing to the treatments with BPI and BSA plus IbeB, and BPI was able to block IbeA-induced apoptosis. Under the same conditions another purified invasion protein IbeB (Huang S H et al.,1999) was unable to induce apoptosis in human EC. Taken together, our data demonstrate that IbeA is a major apoptotic factor in E44 contributing to induction of apoptosis in human EC, which is inhibited by the anti-endotoxin protein BPI (Arditi, M. et al.,. Infect. Immun. 1994, 62:3930-6.). In many brain diseases including bacterial meningitis, neuronal damage or death is due to apoptosis. Blocking IbeA-induced apoptosis by anti-apoptotic agents such as BPI will be a novel approach to prevention and treatment of neonatal E. coli meningitis.

1. A method of providing a complete nucleic acid sequence encoding a gene of a ibeA gene cluster, comprising the steps of:
(a) identifying Escherichia coli structures that contribute to an invasion of BMEC to a ibeA;
(b) extracting and purifying said ibeA from said Escherichia coli structures;
(c) analyzing said extracted and purified ibeA; and
(d) determining a complete nucleic sequence of ibeA.

2. The method, as recited in claim 1, further comprising the steps of
(e) generating a ibeA in-frame deletion mutant;
(f) combining a ibeA in-frame deletion mutant to a ibeA to form a transformants; and
(g) conducting complementation analysis to test an invading ability to BMEC.

3. A probiotic for preventing and treating neonatal meningitis causing meningitic microbes, wherein said probiotic comprises live microorganisms in origin suppressing meningitic virulence factor.

4. The probiotic, as recited in claim 3, wherein said meningitic virulence factor includes GimA.

5. The probiotic, as recited in claim 3, wherein said meningitic microbes are selected from a group consisting E. coli K1 carrying GimA and Group B Streptococcus (GBS).

6. The probiotic, as recited in claim 4, wherein said meningitic microbes are selected from a group consisting E. coli K1 carrying GimA and Group B Streptococcus (GBS).

7. The probiotic, as recited in claim 3, wherein said meningitic microbes are selected from a group consisting of E. coli bacteria, GBS bacteria, Listeria moncytogenes bacteria, Pseudomonas species bacteria, Streptococcus pueumoniae bacteria, Neisseria meningitides bacteria, Haemophilus influenzae bateria, Citrobacter species bacteria, Canida albicans fungus, enteroviruses, herpes simplex viruses, and Toxoplasma gondii parasites.

8. The probiotic, as recited in claim 4, wherein said meningitic microbes are selected from a group consisting of E. coli bacteria, GBS bacteria, Listeria moncytogenes bacteria, Pseudomonas species bacteria, Streptococcus pueumoniae bacteria, Neisseria meningitides bacteria, Haemophilus influenzae bateria, Citrobacter species bacteria, Canida albicans fungus, enteroviruses, herpes simplex viruses, and Toxoplasma gondii parasites.

9. The probiotic, as recited in claim 3, wherein said live microorganisms include bacteria selected from a group consisting of Lactobacillus species, Bifidobacterium species, E. coli Nissle 1917, and probiotic bacteria carrying antigens against one or more virulence factors including GimA.

10. The probiotic, as recited in claim 3, wherein said live microorganisms include yeast in origin suppressing one or more meningitic virulence factors including GimA.

11. A probiotic method for preventing and treating neonatal meningitis caused by meningitic microbes, wherein said probiotic method comprises administering a therapeutically effective prebiotic agents enhancing benefit effects of probiotic organisms and suppressing one or more meningitic virulence factors.

12. The probiotic method, as recited in claim 13, wherein said meningitic virulence factor is GimA.

13. The probiotic method, as recited in claim 11, wherein said prebiotic agents include oligo-saccharides selected from a group consisting of fructooligosaccharides (FOS), inulin, lactulose and galactooligosaccharides.

14. The probiotic method, as recited in claim 12, wherein said prebiotic agents include oligo-saccharides selected from a group consisting of fructooligosaccharides (FOS), inulin, lactulose and galactooligosaccharides.

15. The probiotic method, as recited in claim 11, wherein said prebiotic agents include bacteria selected from a group consisting of Lactobacillus species, Bifidobacterium species, E. coli Nissle 1917, and probiotic bacteria carrying antigens against one or more virulence factors including GimA.

16. The probiotic method, as recited in claim 11, wherein said prebiotic agentsare nondigestible food substances that improve health by stimulating the growth or activity of beneficial bacteria and suppressing said meningitic virulence factors including GimA

17. A probiotic method for treating neonatal meningitis caused by meningitic microbes, comprising a step of administering a therapeutically effective prebiotic agents enhancing benefit effects of probiotic organisms and suppressing one or more meningitic virulence factors.
